(19) United States
(12) Patent Application Publication
(10) Pub. No.: US 2009/0226913 A1
(43) Pub. Date: Sep. 10, 2009

(54) IDENTIFICATION OF CANCER BIOMARKERS AND PHOSPHORYLATED PROTEINS

(75) Inventors: Robert M. Pope, San Marcos, CA (US); Xiquan Liang, Escondido, CA (US); Mahbod Hajivandi, Vista, CA (US); John Leite, Vista, CA (US)

Correspondence Address: LIFE TECHNOLOGIES CORPORATION;C/O INTELLEVATE, P.O. BOX 52050, MINNEAPOLIS, MN, 55402, US

(73) Assignee: LIFE TECHNOLOGIES CORPORATION, Carlsbad, CA (US)

(21) Appl. No.: 12/340,341

(22) Filed: Dec. 19, 2008

Related U.S. Application Data
(63) Continuation of application No. 11/417,264, filed on May 4, 2006.
(60) Provisional application No. 60/678,119, filed on May 4, 2005.
(60) Provisional application No. 60/678,392, filed on May 6, 2005.
(60) Provisional application No. 60/687,355, filed on Jun. 3, 2005.

(51) Int. Cl. C12Q 1/68; C12Q 1/00; G01N 33/574; G01N 33/00

(52) U.S. Cl. 435/6; 435/4; 435/7.23; 436/86; 436/94

(57) ABSTRACT

Methods are provided for identifying proteins that are differentially expressed in disease state and normal cells. Stable isotope labeling of cells in culture allows for the identification of a multiplicity of proteins whose differential abundance in normal and disease state cells can be indicative of the disease state. Biomarkers are identified for breast cancer, in which the biomarkers are proteins having a two-fold or greater difference in abundance between breast cancer and normal cells. Identified biomarkers can be used detection methods that can provide diagnosis, typing, staging, or prognosis of cancer, such as breast cancer, or can be used to predict the response of cancer, such as breast cancer, to one or more anti-cancer agents.

### CROSS-REFERENCE TO RELATED APPLICATIONS

This application claims benefit of priority to U.S. provisional application 60/678,119, filed May 4, 2005, entitled “Cancer Biomarkers” and naming Xiquan Liang, Mahbod Hajivandi, Robert M. Pope, and John Leite as inventors; to U.S. provisional application 60/678,392, filed May 6, 2005, entitled “Identification of Cancer Biomarkers and Phosphorylated Proteins” and naming Robert M. Pope, Xiquan Liang, Mahbod Hajivandi, and John Leite as inventors; and to U.S. provisional application 60/687,355, filed Jun. 3, 2005, entitled “Identification of Cancer Biomarkers and Phosphorylated Proteins” and naming Robert M. Pope, Xiquan Liang, Mahbod Hajivandi, and John Leite as inventors. Each of these applications is incorporated by reference herein in its entirety.

### SEQUENCE LISTING

The instant application contains a “lengthy” Sequence Listing which has been submitted via CD-R in lieu of a printed paper copy, and is hereby incorporated by reference in its entirety. Said CD-R, recorded on May 3, 2006, are labeled CRF, “Copy 1” and “Copy 2”, respectively, and each contains only one identical 1,006 Kb file (IVGN0248.APP).

### BACKGROUND OF THE INVENTION

1. Field of the Invention

The invention relates generally to the identification and detection of molecules that are differentially expressed in different cell types and more specifically to the discovery and use of biomarkers that are indicative of disease states.

2. Background Information

Quantitative proteomics, techniques used to measure differential expression and processing profiles of the entire complement of gene products, may be used to identify and characterize onset disease biomarkers by comparing cell culture samples from both normal and disease states. Stable isotope labeling with amino acids in cell culture (SILAC) is an emerging technology for quantitative proteomics, allowing quantification of the cellular differences between two different states. SILAC methods and applications are described in U.S. Pat. No. 6,391,649 and U.S. Pat. No. 6,642,059, both of which are herein incorporated by reference in their entireties, and in particular for all disclosure of methods of labeling proteins of cells in culture with isotopes and comparing protein levels of the cell cultures using mass spectrometry (MS).

SILAC uses the natural metabolic machinery of the cell to label proteins with either ‘light’ or ‘heavy’ amino acids made with light (standard) or heavy isotopes. Peptides with either light or heavy amino acids are chemically identical and therefore co-migrate in any separation method (such as SDS-PAGE, IEF or other liquid chromatography, etc.), eliminating quantification error due to unequal sampling. However, the peptides are isotopically distinct so that the mass difference between light and heavy peptides is distinguishable by mass spectrometry (MS). Based on the relative intensity of an isotopic peptide pair in MS, differential protein expression and the status of posttranslational modification between two different samples can be quantified. The correlation of a particular peptide to the precursor protein from which it originates is based upon the fragmentation pattern of the (usually mass-selected) peptide, hence its MS/MS profile.

SILAC offers additional advantages over other technologies such as ease of use, compatibility with any lysis buffer or separation technology, and 100% labeling efficiency. Because the incorporation of stable isotopic forms of amino acids occurs as the proteins are assembled or degraded in cell culture, these chemically identical proteins are copies of their light congeners bearing what are, in effect, “label-less labels” at every site of the substituted amino acid. Moreover, since one is free to select any amino acid as a label, one may select an amino acid specific to any protease used in a digestion protocol later and, thus achieve a single label on each and every digest fragment. This makes it possible to track the status of posttranslational modifications, because in principle, proteome coverage is complete.

The present invention expands the use of SILAC to the detection of biomarkers that can be useful in the detection and classification of disease state cells, such as cancer cells. The present invention provides methods of identifying biomarkers using SILAC, methods of detecting and classifying cells using biomarkers identified by SILAC, and biomarkers useful in the detection and classification of cancer, particularly breast cancer.

### SUMMARY OF THE INVENTION

The present invention provides methods of identifying a multiplicity of proteins whose levels differ among cells of a disease state and normal cells using stable isotope labeling of cells in culture, and using one or more of the identified proteins as biomarkers for a disease state. The present invention provides reliable methods of identifying markers that are differentially expressed in one or more cell compartments, such as, for example, cell membranes.

In one aspect, the present invention includes a method of identifying at least one biomarker for cells of a disease state, where the method includes: providing a first cell culture of disease state cells; providing a second cell culture of control cells, in which the cell culture media of the first cell culture contains at least one isotope at a non-natural level in a form that is metabolically incorporated into proteins within cultured cells, in which the isotope is present at a natural level is the second cell culture; allowing the cells in each of the cell cultures to divide; combining at least a portion of the cells of the first cell culture with at least a portion of the cells of the second cell culture to form a mixed cell sample; separating one or more proteins from the mixed cell sample; performing mass spectrometry on the one or more proteins, or peptides generated from the proteins, to obtain a mass spectrometry profile; using the mass spectrometry profile to compare the abundance of at least one of the one or more proteins containing the isotope at a natural level with the abundance of the one or more corresponding proteins that contain the isotope at a non-natural level, in which a difference in abundance of a protein having the isotope at a non-natural level and the same protein having the isotope at a natural level is indicative of a biomarker for disease state cells.

In some preferred embodiments, disease state and normal cells are fractionated prior to the separation of proteins for mass spectrometry. For example, cells can be fractionated for the isolation of cell membranes, mitochondria, nuclei, or cytosolic fractions. Proteins from cell fractions can be further separated, such as by electrophoresis or chromatography, preferably digested with a protease, and analyzed using mass spectrometry to identify proteins that have different abundances in disease state and normal-cells.

The present invention also provides biomarkers for cancer. Such biomarkers can be used to detect or diagnose cancer in a subject, such as a patient known to have or suspected of having cancer. One or more than one of the identified biomarkers can be used to detect, diagnose, type, stage, provide a prognosis for, or predict a drug response of cancer in a patient. One or more than one of the identified biomarkers can be used to detect, diagnose, type, stage, provide a prognosis for, or predict a drug response of breast cancer in a patient.

Data provided herein identifies the proteins of Table 1 as being proteins that are differentially expressed in human breast cancer cells. Accordingly, provided herein is a method of detecting one or more biomolecules, comprising detecting in a biological sample, expression of a protein of Table 1, or a nucleic acid encoding a protein of Table 1, wherein said biological sample is a sample of a patient with a breast pathology. The biological sample can be, for example, a tumor biopsy sample or a breast tumor biopsy sample. Furthermore, the sample can be a fluid sample. For example, sample of blood, plasma, serum, urine, saliva, lymphatic fluid, pelvic lavage, lung aspirate, nipple aspirate, or breast duct lavage.

In certain aspects of the invention, expression is detected of two or more, three or more, four or more, five or more, six or more, seven of more, eight or more, nine or more, ten or more, twenty-five or more, one-half, one-third, or all of the proteins of Table 1, or of nucleic acids encoding two or more, three or more, four or more, five or more, six or more, seven of more, eight or more, nine or more, ten or more, twenty-five or more, one-half, one-third, or all of the proteins of Table 1.

Certain aspects of the invention provide quantitative methods. For example, in certain aspects an expression level is determined of a protein of Table 1, or a nucleic acid encoding a protein of Table 1. In certain embodiments, an altered expression level in the biological sample compared to a normal sample is indicative of the presence of a breast pathology, such as breast cancer. Furthermore, expression levels can be correlated with a type of cancer, a stage of cancer, a prognosis, and/or response to one or more anti-cancer agents.

Typically, methods of this embodiment of the invention are performed by contacting the biological sample with a specific binding reagent that binds to the protein or the nucleic acid. Expression levels can then be quantitated by measuring the amount of specific binding reagent that binds to biomolecules in the sample.

In another embodiment, provided herein is a kit that includes a specific binding reagent that binds to a protein of Table 1, or that binds to a nucleic acid encoding a protein of Table 1. Furthermore, the kits typically include a control that is a biological sample derived from a subject having a breast pathology. For example, the control can include cells obtained directly from a subject having a breast pathology, or tissue culture cells derived from cells of a subject having a breast pathology, such as breast cancer or a breast tumor. The specific binding reagent of the kit is typically an antibody or a nucleic acid. The specific binding reagent is typically present in one or more tubes that are associated together in packaging and shipped from a manufacturer to a customer.

The kit can include additional specific binding reagents. For example, specific binding reagents that bind to one or more, two or more, three or more, four or more, five or more, six or more, seven of more, eight or more, nine or more, ten or more, twenty-five or more, ½, ⅓ or all of the proteins, or encoding nucleic acids of Table 1.

In one embodiment, the kit includes specific binding reagents that bind to one or more, two or more, three or more, four or more, five or more, six or more, seven of more, eight or more, nine or more, ten or more, twenty-five or more, one-half, one-third, or all of the proteins, or encoding nucleic acids of Table 2.

In another aspect the present invention provides methods for identifying one or more phosphoproteins in cells using stable isotope labeling of cells in culture, and identifying one or more phosphoproteins whose abundance differs between or among cells of different types or states, or between or among cells subjected to different treatments, such as different stimuli or inhibitors. One embodiment provides methods of identifying phosphoproteins whose phosphorylation state differs between cells treated with a kinase inhibitor and non-treated cells, where the abundance of a phosphopeptide derived from a phosphoprotein relative to a nonphosphorylated form of the phosphopeptide is indicative of a difference in the phosphorylation state of the phosphoprotein between the compared cells. A related embodiment provides methods of identifying phosphoproteins whose phosphorylation state differs among cells treated with a kinase inhibitor for a different time period or at a different concentration, where the abundance of a phosphopeptide derived from a phosphoprotein relative to a nonphosphorylated form of the phosphopeptide is indicative of a difference in the phosphorylation state of the phosphoprotein between or among the compared cultures.

The present invention also includes a method of isolating phosphoproteins and phosphopeptides from samples, where the method includes: providing a sample that contains one or more phosphoproteins or phosphopeptides; applying the sample to a column that comprises a matrix having a resin derivatized with iminodiacetic acid that has been charged with Fe⁺³; and eluting one or more phosphoproteins or phosphopeptides from the column to obtain one or more isolated phosphoproteins or phosphopeptides. In preferred embodiments, the sample is derived from a cellular fraction and the one or more proteins of the cellular fraction have been digested with at least one protease prior to applying the sample to the column. In this preferred embodiment, the column is used to isolate phosphopeptides.

In yet another aspect, the invention provides a peptide derived from the Dok-2 protein that has a tyrosine residue that is phosphorylated in CML cells, where the peptide comprises the sequence GQEGEYAVPFDAVAR (SEQ ID NO: 186). The peptide spans amino acids 294 through 318 of the human Dok-2 protein (SEQ ID NO:187; Genbank accession no. gi 41406050). The invention includes uses of the Dok-2 peptide in kinase and phosphatase assays. The invention also includes peptides having sequences homologous to the human Dok-2 294-318 sequence that can be phosphorylated by the Bcr-Abl kinase, other kinases that are inhibited by ST1571, such as but in no way limited to sequences of Dok-2 proteins or homologs of other species, or other kinases that are directly or indirectly regulated by the Bcr-Abl kinase or kinases inhibited by ST1571. The invention also includes peptide and proteins comprising the human Dok-2 294-318 sequence, or sequences homologous to the human Dok-2 294-318 sequence, that can be phosphorylated by the Bcr-Abl kinase, other kinases that are inhibited by ST1571, or other kinases that are directly or indirectly regulated by the Bcr-Abl kinase or kinases inhibited by ST1571.

### BRIEF DESCRIPTION OF THE DRAWINGS

FIG. 1 is a schematic depiction of one embodiment of the present invention for relative quantification of membrane proteins between normal and malignant breast cell lines originating from the same patient with breast carcinoma.

FIG. 2 depicts the precision of quantification of peptides by SILAC/MS. The tryptic peptide extract with light and heavy labels was analyzed by RPLC with Q-TOF detection. Multiple isotopic peptide pairs derived from vimentin were recovered in a single LC-MS/MS run and were used to calculate the precision of quantification.

FIG. 3 depicts MS spectra that demonstrate the reproducibility of SILAC experiments. SILAC experiments were performed three times separately. The top panel shows the spectra for a vimentin peptide and the bottom panel shows the spectra for an oxygen regulated protein peptide detected in the three experiments.

FIG. 4 depicts MS spectra that demonstrate that the relative ratio of isotopic peptide pairs is consistent regardless of the charge state of peptides as well as the number of Lys and/or Arg contained in the peptides.

FIG. 5 is a Western blot of normal (N) and cancerous (C) breast cells grown in culture with antibodies against osteoblast-specific factor 2 (upper panel), DNA-activated protein kinase (middle panel), and alpha 2 macroglobulin (lower panel). (S) designates culture medium of breast cancer cells, in which osteoblast-specific factor 2 was detected.

FIG. 6 depicts mass spectra of phosphopeptides from tryptic digests of beta-casein isolated with A) a commercially available phosphopeptide isolation kit from Pierce, or B) Toyopearl resins.

FIG. 7(A) depicts the mass spectrum of a phosphopeptide of m/z 1688.7 from Dok-2 tryptic digest using IMAC. (B) provides the MS/MS spectrum of doubly charged 845.44 m/z precursor from Dok-2 analyzed on the Waters Q/TOF API US

FIG. 8 shows MALDI-TOF mass spectra of Dok-2 peptide isolated from heavy tyrosine-labeled cells with and without one hour (A) or two hours (B) of kinase inhibitor treatment.

FIG. 9 shows MALDI-TOF mass spectra of cells labeled with heavy tyrosine and in the presence and absence of a kinase inhibitor that allows identification of phosphoryated peptides.

### DETAILED DESCRIPTION OF THE INVENTION

The present invention is based in part on the discovery that biomarkers can be identified by using SILAC to analyze changes in membrane and phosphorylated protein expression in disease state cells, including primary cultured cells. Furthermore, the present invention is based in part on the identification of a set of new breast cancer biomarkers, using the methods provided herein. Additionally, the present invention is based in part on the discovery that an iminodiacetic acid resin is a particularly effective column for phosphoprotein and phosphopeptide isolation, especially in the presence of iron.

### I. SILAC for Identification of Biomarkers

The present invention provides a method of identifying at least one biomarker for cells of a disease state by comparing the levels of one or more proteins in two or more cell cultures using stable isotope labeled amino acids in cell culture. The method comprises: (1) providing a first cell culture comprising disease state cells; (2) providing a second cell culture comprising normal cells, in which the cell culture media of the first cell culture comprises at least one isotope at a non-natural abundance, and the second cell culture does not comprise the at least one isotope at a non-natural abundance, where the isotope at a non-natural abundance is in a from that is metabolically incorporated into proteins within cultured cells; and (3) allowing the cells in each of the cell cultures to divide. After the cells have divided in culture so that isotopes in the culture media have been incorporated into proteins, the method further comprises: (4) combining at least a portion of the cells, cellular constituents, or media of the first cell culture with at least a portion of the cells, cellular constituents, or media of the second cell culture to form a mixed cell sample; (5) separating one or more proteins from the mixed cell sample; (6) performing mass spectrometry on the one or more proteins, or peptides derived from one or more proteins, to obtain a mass spectrometry profile; and (7) using the mass spectrometry profile to compare the level of at least one of the one or more proteins comprising the isotope at a non-natural abundance with the level of the at least one of the one or more proteins that does not comprise the isotope at a non-natural abundance, in which a difference in the level of the non-naturally occurring isotope form of a protein from the naturally occurring isotope form is indicative of a biomarker for cells of a disease state.

Preferably, the second culture comprises an isotope at a much lower abundance than is present in the first culture, for example, the first culture can comprise an isotope at a level that is much greater than its natural abundance, and the second culture can comprise the isotope at a level that is equivalent to the isotopes natural abundance, or can 10% or less of its level in the first cell culture, 5% or less of its level in the first cell culture, 2% or less of its level in the first cell culture, or 1% or less of its level in the first cell culture. In some embodiments of the invention, the first cell culture can comprise an isotope at a level that is greater than its natural abundance, and the second cell culture can comprise the isotope at a level that is not detectable such as by mass spectrometry to detect incorporation of isotopes into protein.

The methods of the present invention compare protein levels of two or more cell cultures. In preferred embodiments, the methods are performed using two cell cultures that preferably comprise cells of the same type, where one culture comprises normal cells and the other culture comprises cells of a disease state. In some preferred applications of the method, the cells can be normal and cancer cells of the same type (for example, normal breast cells and breast cancer cells). Cells can also be of the same type, in which one culture of the cells receives a treatment that induces a disease or pathologic state before or during the culturing of cells with isotope label. For example, one cell culture can be infected with a virus while a corresponding culture of the same type is uninfected. In another example, one cell culture can be treated with one or more agents that causes oxidative damage to cells, while a corresponding culture of the same cell type is untreated.

The methods of the present invention can also be used to identify biomarkers for cell differentiation. In these methods, two or more cultures of stem cells can be compared by growing the cultures in different abundances of heavy isotopes and allowing the heavy isotopes to be incorporated into biomolecules such as proteins. The different cultures can be subjected to different conditions. For example, one culture may be treated with one or more growth factors while another culture is grown in the absence of a growth factor. Cells or cellular components of the cultures can be combined and the proteins or other biomolecules can be analyzed by MS to determine the relative abundances of biomolecules in the cultures. Proteins that differ in abundance in stem cells in response to a growth factor or other stimulus can be used to identify proteins that function in the differentiation pathway.

The method can also be performed for identification of nonprotein modification of proteins (such as, but not limited to, phosphorylation, glycosylation, prenylation, or acylation), or for identification of nonprotein biomarkers, for example, for the identification of carbohydrates, lipids, steroids, or nucleic acids that may differ in abundance between cells of a disease state and normal cells. In these cases, precursor molecules that are to be labeled with one or more heavy isotopes and added to a cell culture are preferably direct or indirect precursors of the groups or molecules whose abundance is to be compared, and can include, for example, glucose, sucrose, one or more nucleotides, phosphate, inositol, choline, isoprenylated acids, econosoic acids, glycerol, cholesterol, etc.

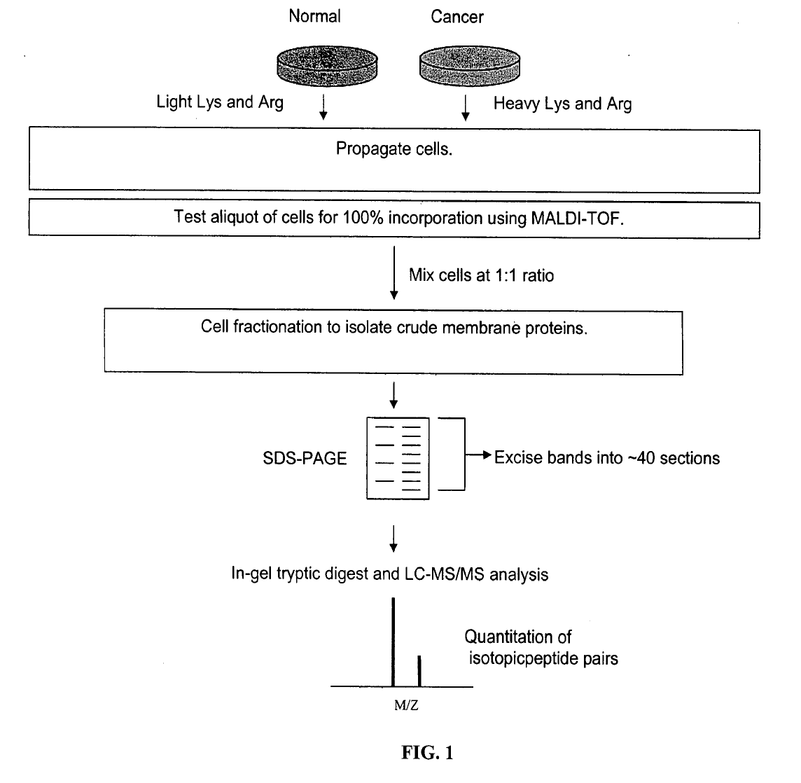

FIG. 1 outlines a protocol for applying the methods of the present invention to identifying biomarkers in cancer cells. In this depiction, normal and cancer cells are grown in media containing either “light” lysine (Lys) and arginine (Arg), that have naturally occurring isotopes of, for example nitrogen and carbon, or “heavy” Lys and Arg, that have a higher abundance of an isotope of nitrogen or carbon, for example, than the natural abundance. The two cultures are incubated for at least six doubling times and then combined at 1:1 ratio. The cell mixture is lysed in hypotonic buffer and crude membranes are obtained by centrifugation. Membrane pellets are dissolved in SDS sample buffer and analyzed by SDS-PAGE. The entire gel lane is divided into approximately 40 sections, followed by in-gel tryptic digestions. Peptide extracts are analyzed by mass spectrometry (here, nanoelectrospray LC-MS/MS). Relative quantification is achieved via the ratios of unique isotopic peptide pairs in the MS spectrum.

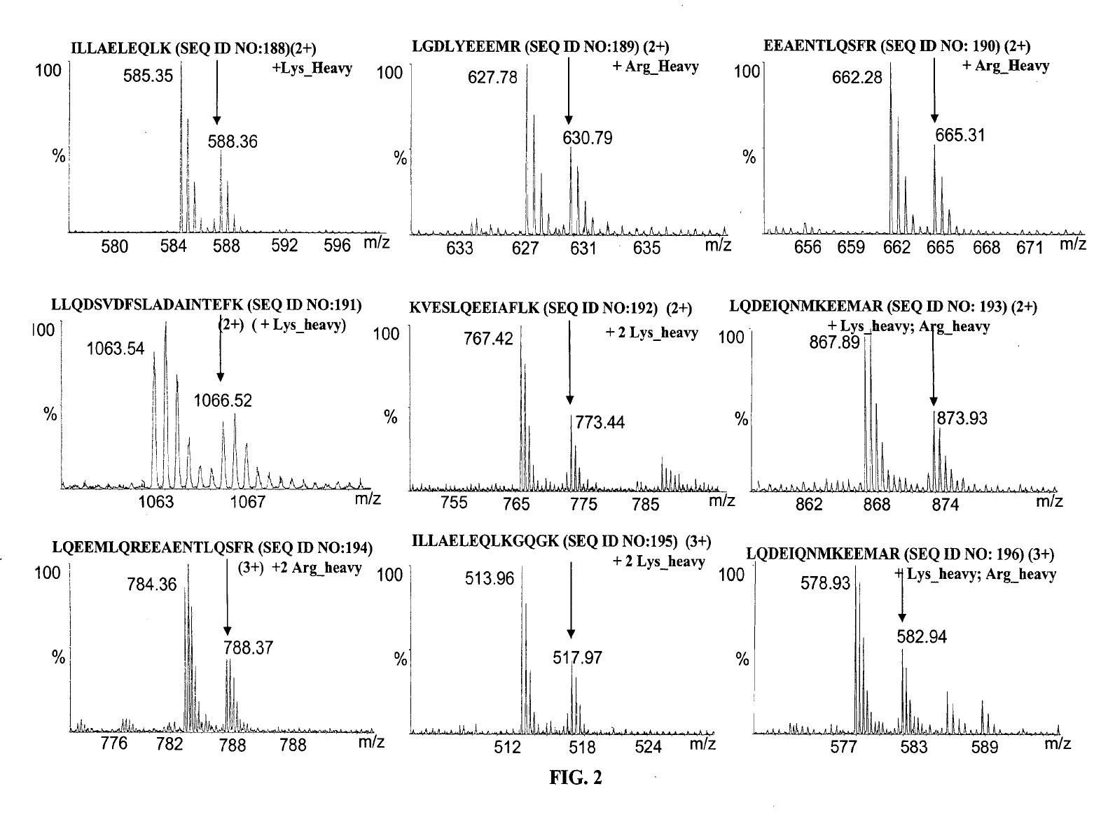

FIG. 2 shows quantitation of peptides by SILAC/MS. In this experiment tryptic peptide extract with light and heavy labels was analyzed by RPLC with Q-TOF detection. Multiple isotopic peptide pairs derived from human vimentin (Genbank gi 418249; SEQ ID NO:43) were recovered in a single LC-MS/MS run and were used to calculate the precision of quantification. The relative ratio of isotopic peptides is remarkably consistent with a standard deviation of ±10%. The relative ratio of isotopic peptide pairs is identical regardless of the charge state of peptides as well as the number of Lys and/or Arg containing in the peptides.

Cells

The cells used in the methods of the present invention can be prokaryotic or eukaryotic cells of any type, and are preferably animal cells, and more preferably are mammalian cells. Cells used in the methods of the present invention are most preferably human cells.

The cells used for identifying biomarkers can be from cell lines or primary cells. The inventors have found that using the methods of the present invention, it is possible to use as few as 10⁶ cells to identify differential expression between two cell types or two cell states. For optimal labeling of cells, the cells are preferably grown in heavy isotope media for at least six doublings to allow for greater than 98% incorporation of label. Thus, starting cultures can have as few as 2×10⁵ cells. This allows for the use of primary cells, such as cells from lines that have not been immortalized through genetic manipulation or have not otherwise become growth factor-independent. This also allows for the use of primary cell isolated directly from a subject, such as cells from tissue samples (including biopsy samples) in which the cell number is limited, to determine differences in protein expression in normal and disease state cells using SILAC methods. Primary cells can be taken from biopsied or sampled tissue or bodily fluids, and are preferably at least partially purified away from other sample components, including other cell types (“nontarget cells”), using, for example, the use of separation steps such as filtration, centrifugation, or selective precipitation; dissection of tissue (including but not limited to laser capture microdissection); affinity separation of components (such as by “panning” using affinity reagents such as antibodies directly or indirectly bound to a solid support such as beads to either remove undesirable sample components or enrich cells of interest); or the application of drugs or reagents to a culture that discourage the growth of nontarget cells.

In cases where primary ceHs are used, proteins expressed by primary disease-state cells can be compared in SILAC experiments with proteins expressed normal cells of a cell line, but preferably are compared with primary normal cells. The primary normal cells can be from the same or a different individual. Proteins expressed by primary normal cells can also be compared with proteins expressed by disease state cells of a cell line.

Isotopic Label

The isotopic labels used in the methods of the present invention can be any molecule that can be metabolically incorporated into protein within cells that has a non-natural abundance of one or more isotopes. As nonlimiting examples, heavy isotopes of carbon, nitrogen, oxygen, hydrogen, and sulfur that are of very low abundance in nature can be highly enriched in molecules used to label proteins (such as amino acids) such that, using mass spectrometry, proteins or peptides that have incorporated the heavy isotopes can be distinguished from corresponding proteins or peptides that have incorporate isotopes of the same element at their natural abundance, that is, “light” isotopes of the element.

Preferred labels are amino acids having non-naturally occurring levels of heavy isotopes, such as, but not limited to, carbon-13, nitrogen-15, oxygen-17, oxygen-18, sulfur-34, and hydrogen-2. An amino acid can be labeled with more than one isotope at a non-natural abundance, for example, an amino acid used in SILAC can have both carbon-13 and nitrogen-15. For a given cell labeling/MS detection experiment, one or more amino acids can be labeled with a non-natural abundance of one or more isotopes. In some preferred embodiments of the present invention, for example, Arg and Lys have incorporated heavy isotopes (such as ¹³C and/or ¹⁵N), and proteins are digested with trypsin prior to mass spectrometry. In these embodiments, each trypsin fragment of a protein of the labeled cell culture (with the exception of carboxy-terminal peptides) comprises a heavy isotope label.

In the methods of the present invention, protein levels of two cultures are compared by comparing heavy isotope-labeled proteins or peptides of one culture with light isotope containing proteins or peptides of the other culture. Preferably, in these methods, one of the cultures comprises a heavy isotope label, where the heavy isotope is at a non-natural abundance, and the other culture does not comprise an isotope label at a non-natural abundance. For example, the first culture can comprise in the media a metabolic precursor molecule that can become incorporated into biomolecules such as proteins within cells, in which the metabolic precursor molecule comprises an isotope at a non-natural abundance, and the second culture can comprise in the media the same metabolic precursor molecule, in which the precursor molecule does not comprise an isotope at a non-natural abundance. The metabolic precursor molecule can be, as nonlimiting examples, a sugar or amino acid. The isotope present at a non-natural abundance can be, for example a heavy isotope.

The heavy isotope label can be present in either the control cell culture or in the culture of disease state cells. In preferred embodiments in which the label is an amino acid that comprises one or more heavy isotopes, preferably the heavy isotope amino acid comprises essentially all of the amino acid in the cell culture to be labeled with heavy isotope. For example, where the label is ¹³C-Arg, preferably the culture media of the culture to be labeled contains ¹³C-Arg to the exclusion of ¹²C-Arg. Preferably, the cell culture that does not comprise a heavy isotope label at a non-natural abundance does not contain a detectable level of the heavy isotope.

Culturing Cells

Normal and disease state cells (or two cultures of stem cells to receive different treatments) are preferably cultured in parallel, in which either the normal cell culture media or the disease state cell culture media comprises a label in the form of an isotope at non-natural abundance. In some preferred embodiments, the normal cell culture media comprises a label in the form of an isotope at non-natural abundance, and the disease state culture media does not comprise a label in the form of an isotope at non-natural abundance. In other preferred embodiments, the disease state cell culture media comprises a label in the form of an isotope at non-natural abundance, and the normal cell culture media does not comprise a label in the form of an isotope at non-natural abundance. In other preferred embodiments, both cell cultures whose cells are being compared comprise isotopic label, where one cell culture comprises a first isotopic label, and the second cell culture comprises a second isotopic label.

In other embodiments, cells can be removed from a tissue, such as but not limited to a cancerous tissue such as a tumor, and grown in culture with an isotopic label. The cells can be combined with cells of the original tumor from patient biopsy for MS analysis to compare the abundance of proteins expressed by tumor cells grown in culture with the abundance of the same proteins expressed by tumor cells in the body. The comparison can be used to identify biomarkers for tumor cells that relate to the ability of the tumor to survive and grow within the body of a patient, such as but not limited to biomarkers that participate in the interaction of cancer cells with normal cells, such as biomarkers related to, for example, tissue infiltration, tumor vascularization, and nutrient procurement. Such biomarkers can be candidate drug targets.

Thus, the present invention provides a method of identifying proteins that enable, mediate, or facilitate tumor growth in the body. The method includes: providing a culture comprising cancer cells removed a tumor from a patient; allowing the tumor cells in the cell culture to divide in media that comprises at least one isotope at a non-natural level in a form that is metabolically incorporated into proteins within cultured cells; combining at least a portion of the cells of the cell culture with a sample of cells taken directly from the tumor to form a mixed cell sample; separating one or more proteins from the mixed cell sample; performing mass spectrometry on the one or more proteins, or peptides thereof, to obtain a mass spectrometry profile; and using the mass spectrometry profile to compare the abundance of at least one of the one or more proteins comprising the non-naturally occurring isotope with the abundance of the at least one of the one or more proteins that does not comprise the non-naturally-occurring isotope. In these methods, a difference in the abundance of the non-naturally occurring isotope form relative to the naturally occurring isotope form is indicative of a protein that enables, facilitates, or mediates the growth of tumor cells in the body.

Stem cells or progenitor cells can also be removed from an animal and grown in culture with an isotopic label. After growth in culture to incorporate label, the cells, or components of the cells, can be combined with cells or cell components taken directly from the animal for MS analysis to compare the abundance of proteins expressed by stem cells grown in culture with the abundance of the same proteins expressed by stem cells in the developing organism. The comparison can be used to identify biomarkers for stem cell differentiation, such as biomarkers related to in vivo cell-cell interaction, in vivo cell-substrate interaction, or in vivo response to cytokines. Such biomarkers can be used to dissect the differentiation pathway.

In these aspects, the invention provides methods for identifying proteins that mediate the differentiation of cells in the body. The methods include: providing a culture comprising stem cells removed a developing animal; allowing the stem cells in the cell culture to divide in media that comprises at least one isotope at a non-natural level in a form that is metabolically incorporated into proteins within cultured cells; combining at least a portion of the cells of the cell culture with a sample of cells taken directly from the animal to form a mixed cell sample, wherein the cells taken directly from the animal are derived from stem cells from the same type as the stem cells in the cell culture; separating one or more proteins from the mixed cell sample; performing mass spectrometry on the one or more proteins, or peptides thereof, to obtain a mass spectrometry profile; using the mass spectrometry profile to compare the abundance of at least one of the one or more proteins comprising the non-naturally occurring isotope with the abundance of the at least one of the one or more proteins that does not comprise the non-naturally-occurring isotope, wherein a difference in abundance of the non-naturally occurring isotope form from the naturally occurring isotope form is indicative of a protein that enables, facilitates, or mediates the differentiation of stem cells in the body.

Preferably the label is a heavy isotope of, for example, carbon, nitrogen, sulfur, oxygen, or hydrogen that is incorporated into an amino acid in the cell culture media used for labeling cells. Preferably the cell culture media used for labeling cells comprises one or more heavy isotope-labeled amino acids, in which greater than 95%, and even more preferably greater than 98%, of the one or more amino acids that are labeled comprise the heavy isotope.

For example, cells that are to be labeled with one or more particular amino acids that have one or more incorporated heavy isotopes can be grown in DMEM, RPMI, or any other suitable media to which the one or more heavy isotope amino acids have been added. Parallel cultures in which the proteins are not to be labeled with heavy isotope amino acids preferably are supplemented with the same amino acids as the labeled cultures, but in this case the amino acids do not comprise heavy isotopes. Preferably, the media and media supplements used to culture the cells do not contain the particular amino acids that are to be supplied to the cultures in heavy isotope form for labeling. For example, serum used for culturing the cells should be dialyzed to remove amino acids.

As nonlimiting examples, cells to be labeled can be grown in DMEM or RPMI medium to which dialyzed FBS has been added to a final concentration of 10%. The media can be supplemented with glutamine (where glutamine is not used as a labeled amino acid), and, optionally, pencillin and/or streptomycin at standard concentrations. Purified growth factors or cytokines can be supplemented to the media if they are essential to cell growth or desirable for the experiment being performed.

In these examples, the labeling media also contains 100 mg of at least one heavy isotope amino acid, such as, for example, [U-¹³C₆] L-Lysine, [U-¹³C₆] L-Arginine, or [U-¹⁵N₄, U-¹³C₆,] L-Arginine. (In one preferred embodiment, the media contains 100 mg/liter heavy Lys ([U-¹³C₆] L-Lysine) and 100 mg/liter heavy Arg ([U-¹⁵N₄, U-¹³C₆,] L-Arginine), such that peptides containing heavy Lys experience a shift of 6 Da relative to their unlabeled counterparts, and peptides containing heavy Arg experience a shift of 10 Da relative to their unlabeled counterparts.) The corresponding non-labeling media is supplemented with the same amounts of the same amino acids as the labeling media, but in this case in their light isotope form. For example, where the labeling media is supplemented with 100 mg per liter of each of heavy Arg and heavy Lys, the non-labeling media is supplemented with 100 mg per liter of each of non-heavy isotope Arg and non-heavy isotope Lys.

Cell are preferably grown in labeling media for at least six doublings. For example, a starting culture of 10⁵ cells can be grown to a final cell number of 6.4×10⁶ cells. This ensures essentially 100% incorporation of heavy isotope amino acids into proteins of the cells. Depending on the growth rate of the cells and the culture density, the cells can be split with light or heavy labeling medium separately.

To ensure 100% incorporation of heavy amino acids into proteins, small aliquots of cells (10⁵-10⁶) labeled with light or heavy amino acids can be removed and lysed separately in 500 μl of SDS sample buffer and analyzed by SDS-PAGE side by side. One or two protein bands are picked randomly, excised from the gel side by side, and subjected to in-gel tryptic digest, followed by the MALDI-TOF analysis. Alternatively, cells (10⁷) labeled with light or heavy amino acids can be lysed in cell lysis buffer separately. Proteins of interest can then be immunoprecipitated from the cell lysates and analyzed by SDS-PAGE side by side. Protein bands are excised, digested with trypsin, and then analyzed by MALDI-TOF in parallel. Compared to peptides labeled with light amino acids, peptides labeled with heavy amino acids should increase a few Daltons in mass depending on the nature of the heavy amino acids used for labeling (e.g. 6 Da for ¹³C labeled Lys and 10 Da for ¹³C, ¹⁵N double-labeled Arg). If only heavy ¹³C Lys is used for labeling, only peptides containing a Lys residue will shift 6 Da in mass, but peptides containing an Arg residue would have the same mass. In this way, before proceeding with an experiment, close to 100% incorporation of heavy amino acid into peptides can be verified, which means that no or a very little of corresponding light peptides should be detected in a peptide sample from cells labeled with heavy amino acids.

Mixing of Samples

In most applications of the method control cells and a disease state cells are mixed prior to performing any cell fractionation or protein separation steps. The mixing of control and disease state cells prior to further manipulation avoids sample-to-sample variation in the downstream steps leading to mass spectrometry that can lead to error in calculating relative abundances of proteins in the cultures being compared. In these procedures, aliquots of the two cell cultures being compared are preferably counted and equal numbers of control and disease-state cells are mixed together to form a mixed cell sample.

However, it may be desirable in some circumstances to mix cell organelles, cell lysates, extracts, or fractions after control and normal cell cultures have been separately lysed, and, optionally, subjected to one or more fractionation or separation steps. In these cases, equal amounts of the lysates, extracts, or fractions can be mixed after quantitating or measuring activity of one or more cellular components.

In methods of the present invention in which cell media is analyzed to detect secreted proteins, the cell supernatants are mixed. Preferably, aliquots of each cell culture are counted so that the amount of cell supernatants of the two cell cultures that are mixed together is standardized to the number of cells in each culture. It is also possible in this case to mix cell supernatants of each cell culture based on equal protein content of the cell supernatants, or equal amounts or activities of one or more components the cell supernatants.

Fractionating Cells/Separation of Proteins

To reduce the complexity of proteins subjected to MS and focus the search for differentially expressed proteins to a particular type of protein or cell compartment, it is generally desirable to fractionate cells, separate proteins, or both, prior to performing mass spectrometry.

In some embodiments of the present invention, a biomarker can be identified as a biomolecule in which the abundance of the biomarker in a given cell compartment or fraction differs between disease state and normal cells, or between cultures of disease state cells or stem cells that receive different treatments. For example, as described in Example 1, osteoblast specific factor 2 (OSF-2; Genbank gi 46576887, SEQ ID NO:79), a biomarker identified using the methods of the present invention, is detected in the nucleus of noncancerous breast cells and on the cell membranes of breast cancer cells.

Depending on the cell compartment to be analyzed, after harvesting, cells can be lysed in different buffers or solutions. Preferably the lysis buffers comprise protease inhibitors to preserve protein integrity and nucleases to remove nucleic acids. Cell lysis buffers and solutions can vary in their components and can be optimized for the type of protein or cell compartment to be analyzed using SILAC/MS. For example, to analyze abundances of membrane proteins, cells can be lysed in a buffer containing a hypotonic buffer. One example of a hypotonic lysis buffer is 10 mM Tris, pH 7.4, 1 mM MgCl₂, 10 units per milliliter benzonase, 0.5 mM PMSF, 0.15 micromolar aprotinin, and 1 micromolar leupeptin hemisulfate. For the isolation of cytosolic proteins, cells can optionally be lysed in buffers containing nonionic detergents, ionic detergents, or a combination thereof. One example of a buffer for cell lysis contains 50 mM Tris-HCl, pH 8, 1% NP-40, 150 mM NaCl, 1 mM Na₃VO₄, 10 mM NaF, 0.15 micromolar aprotinin, and 1 micromolar leupeptin hemisulfate. Another example of a buffer for cell lysis contains 50 mM Tris-HCl, pH 8, 1% TritonX-100, 0.5% deoxycholate, 0.1% SDS, 500 mM NaCl, 1 mM Na₃VO₄, 10 mM NaF, 0.15 micromolar aprotinin, and 1 micromolar leupeptin hemisulfate.

Cell fractionation can be used to investigate different abundance of proteins of a cell compartment of interest, such as, for example, the cytosol, the nucleus, the mitochondria, cell membranes, cytoskeleton, etc. Cell fractionation methods are well known in the cell biology, and can include zonal or gradient centrifugation, selective lysis of membrane components, filtration, etc.

Many membrane proteins mediate the response of the cell to external factors, such as growth factors, hormones, other cells, and cell substrates. Therefore, the separation of cell membranes for investigating differences in abundance of membrane proteins between normal cells and disease state cells is of particular interest. In addition to serving as biomarkers, membrane proteins identified as being differentially expressed in a disease state can be candidate drug targets.

Methods of isolating cell membranes are well-known in cell biology. Cell membrane isolation can be performed, for example, by hypotonic lysis of cells and removal of nuclei by centrifugation. Cell membranes (or microsomes) can be recovered by ultracentrifugation. The pelleted membranes can be solubilized in a detergent-containing buffer, and the proteins can be separated using PAGE.

In some cases it may be desirable to isolate peripheral membrane proteins. In this case cell membranes isolated by ultracentrifugation can be salt-extracted or treated with low concentrations of mild detergents to remove membrane-associated proteins.

In some methods of the present invention, the cell media is analyzed for secreted proteins. In these embodiments, the cell cultures are harvested, equivalent amounts of each of the cultures are mixed (for example, based on cell counts), and cells are pelleted. The cell media supernatant is recovered for enrichment of secreted proteins. Proteins of the supernatant can be concentrated, for example by ultrafiltration or dialysis.

Proteins isolated from cell media or cell fractions can be separated to reduce the complexity of the samples analyzed by mass spectrometry. Separation of proteins can be, for example, by affinity capture, selective solubilization, selective precipitation, chromatography, or electrophoresis. Affintiy capture can be used to separate a single protein or protein family, epitope tagged proteins, or a broad class or proteins, such as, for example, proteins containing phosphotyrosine. Chromatography can be affinity chromatography, or can separate proteins based on size, charge, or hydrophobicity. Chromatographic separation can be coupled to mass spectrometry, as described below, to sequentially analyze fractions as they elute from a column matrix. Electrophoresis, such as PAGE, can be used to separate proteins based on size. PAGE can be under denaturing or nondenaturing conditions, or two-dimensional PAGE can be performed. After electrophoresis, a gel lane comprising separated proteins can be divided into slices. Proteins extracted from each of the slices or any subset of the slices can be analyzed separately using mass spectrometry.

For identification of proteins using SILAC, the proteins are preferably digested into peptides prior to mass spectrometry. Proteins can be digested using any protease or chemical peptide cleavage reagent that generates peptides of from about 5 to about 200 amino acids. Examples of proteases that can be used include trypsin, V-8 protease, pepsin, subtilisin, proteinase 1c, and tobacco etch virus protease. Cyanogen bromide can also be used. Trypsin is preferred in some embodiments of the present invention in which arginine and lysine are isotopically labeled amino acids. In these embodiments, because trypsin digests proteins C-terminal to arg and lys residues, each trypsin fragment (except for carboxy terminal fragments of proteins) will have an isotopic label.

A general tryptic digest protocol is as follows:

To avoid contamination with keratin, gel manipulation and digestion steps are performed in a laminar flow hood. Protein bands of interest are excised, along with positive and negative controls, and at least one blank from the SDS-PAGE gel. NuPAGE® Novex® acrylamide gels, available from Invitrogen (Carlsbad, Calif., USA) are recommended for protein separation. Each band/or spot is chopped into small (approx 1 mm diameter) particles with a clean pipette tip. The gel pieces are transferred to a clean microcentrifuge tube. Microtubes from Axygen (Union City, Calif.) or Eppendorf (Hamburg, Germany) are recommended. The gel pieces are destained two to three times with 40% acetonitrile in 25 mM NH₄HCO₃ (pH 8.0) until no blue hue is observed. The gel pieces are dehydrated with 100% acetonitrile and dried for 5 min with a Speed Vac lyophilizer (Savant).

About 10 μl of cold 10 ng/ml proteomics-grade trypsin in 25 mM NH₄HCO₃ (pH 8.0) is added to the dried gel pieces. The gel pieces are incubated on ice for 1 to 2 hr as they swell to minimize auto proteolysis as the trypsin soaks into the gel. Just enough trypsin solution is added to cover the gel pieces. The microcentrifuge tube is covered with aluminum foil to ensure uniform heating, and the gel pieces are incubated overnight at 37° C.

Approximately 30 μl of 1.5% TFA is added to the tryptic digestion mix and incubated at room temperature for about 30 min. The tubes are vortexed about 1 min, centrifuged briefly, and the peptide extract is transferred into a clean tube (Axygen or Eppendorf microcentrifuge tubes are recommended). The gel pieces are further extracted with 30 to 50 μl of 50% acetonitrile in 0.75% TFA for 30 min. and vortexed for about 1 min. The peptide extracts are combined and analyzed by MALDI-TOF. For LC-ESI/MS analysis, the peptide extract should be dried under vacuum and the peptides resuspended in 20 μl of 10% acetonitrile in 0.1% formic acid. Alternatively, 2% formic acid can be used in place of TFA in the steps above.

Mass Spectrometry

In various aspects, the invention is drawn to mass spectroscopy. As used herein, the term “mass spectrometry” (or simply “MS”) encompasses any spectrometric technique or process in which molecules are ionized and separated and/or analyzed based on their respective molecular weights. Thus, as used herein, the terms “mass spectrometry” and “MS” encompass any type of ionization method, including without limitation electrospray ionization (ESI), atmospheric-pressure chemical ionization (APCI) and other forms of atmospheric pressure ionization (API), and laser irradiation. Mass spectrometers are commonly combined with separation methods such as gas chromatography (GC) and liquid chromatography (LC). GC or LC separates the components in a mixture, and the components are then individually introduced into the mass spectrometer; such techniques are generally called GC/MS and LC/MS, respectively. MS/MS is an analogous technique where the first-stage separation device is another mass spectrometer. In LC/MS/MS, the separation methods comprise liquid chromatography and MS. Any combination (e.g., GC/MS/MS, GC/LC/MS, GC/LC/MS/MS, etc.) of methods can be used to practice the invention. In such combinations, “MS” can refer to any form of mass spectrometry; by way of non-limiting example, “LC/MS” encompasses LC/ESI MS and LC/MALDI-TOF MS. Thus, as used herein, the terms “mass spectrometry” and “MS” include without limitation APCI MS; ESI MS; GC MS; MALDI-TOF MS; LC/MS combinations; LC/MS/MS combinations; MS/MS combinations; etc.

HPLC and RP-HPLC

It is often necessary to prepare samples comprising an analyte of interest for MS. Such preparations include without limitation purification and/or buffer exchange. Any appropriate method, or combination of methods, can be used to prepare samples for MS. One preferred type of MS preparative method is liquid chromatography (LC), including without limitation HPLC and RP-HPLC.

High-pressure liquid chromatography (HPLC) is a separative and quantitative analytical tool that is generally robust, reliable and flexible. Reverse-phase (RP) is a commonly used stationary phase that is characterized by alkyl chains of specific length immobilized to a silica bead support. RP-HPLC is suitable for the separation and analysis of various types of compounds including without limitation biomolecules, (e.g., glycoconjugates, proteins, peptides, and nucleic acids, and, with mobile phase supplements, oligonucleotides). One of the most important reasons that RP-HPLC has been the technique of choice amongst all HPLC techniques is its compatibility with electrospray ionization (ESI). During ESI, liquid samples can be introduced into a mass spectrometer by a process that creates multiple charged ions (Wilm et al., Anal. Chem. 68:1, 1996). However, multiple ions can result in complex spectra and reduced sensitivity.

In HPLC, peptides and proteins are injected into a column, typically silica based C18. An aqueous buffer is used to elute the salts, while the peptides and proteins are eluted with a mixture of aqueous solvent (water) and organic solvent (acetonitrile, methanol, propanol). The aqueous phase is generally HPLC grade water with 0.1% acid and the organic solvent phase is generally an HPLC grade acetonitrile or methanol with 0.1% acid. The acid is used to improve the chromatographic peak shape and to provide a source of protons in reverse phase LC/MS. The acids most commonly used are formic acid, trifluoroacetic acid, and acetic acid. In RP HPLC, compounds are separated based on their hydrophobic character. With an LC system coupled to the mass spectrometer through an ESI source and the ability to perform data-dependant scanning, it is now possible in at least some instances to distinguish proteins in complex mixtures containing more than 50 components without first purifying each protein to homogeneity. Where the complexity of the mixture is extreme, it is possible to couple ion exchange chromatography and RP-HPLC in tandem to identify proteins from mixtures containing in excess of 1,000 proteins.

MALDI-TOF MS

A particular type of MS technique, matrix-assisted laser desorption time-of-flight mass spectrometry (MALDI-TOF MS) (Karas et al., Int. J. Mass Spectrom. Ion Processes 78:53, 1987), has received prominence in analysis of biological polymers for its desirable characteristics, such as relative ease of sample preparation, predominance of singly charged ions in mass spectra, sensitivity and high speed. MALDI-TOF MS is a technique in which a UV-light absorbing matrix and a molecule of interest (analyte) are mixed and co-precipitated, thus forming analyte:matrix crystals. The crystals are irradiated by a nanosecond laser pulse. Most of the laser energy is absorbed by the matrix, which prevents unwanted fragmentation of the biomolecule. Nevertheless, matrix molecules transfer their energy to analyte molecules, causing them to vaporize and ionize. The ionized molecules are accelerated in an electric field and enter the flight tube. During their flight in this tube, different molecules are separated according to their mass to charge (m/z) ratio and reach the detector at different times. Each molecule yields a distinct signal. The method is used for detection and characterization of biomolecules, such as proteins, peptides, oligosaecharides and oligonucleotides, with molecular masses between about 400 and about 500,000 Da, or higher. MALDI-MS is a sensitive technique that allows the detection of low (10⁻¹⁵ to 10⁻¹⁸ mole) quantities of analyte in a sample.

Partial amino acid sequences of proteins can be determined by enzymatic proteolysis followed by MS analysis of the product peptides. These amino acid sequences can be used for in silico examination of DNA and/or protein sequence databases. Matched amino acid sequences can indicate proteins, domains and/or motifs having a known function and/or tertiary structure. For example, amino acid sequences from an uncharacterized protein might match the sequence or structure of a domain or motif that binds a ligand. As another example, the amino acid sequences can be used in vitro as antigens to generate antibodies to the protein and other related proteins from other biological source material (e.g., from a different tissue or organ, or from another species). There are many additional uses for MS, particularly MALDI-TOF MS, in the fields of genomics, proteomics and drug discovery. For a general review of the use of MALDI-TOF MS in proteomics and genomics, see Bonk et al. (Neuroscientist 7:12, 2001).

Tryptic peptides labeled with light or heavy amino acids can be directly analyzed using MALDI-TOF. However, where sample complexity is apparent, on-line or off-line LC-MS/MS or two-dimensional LC-MS/MS is necessary to separate the peptides. For example, for simple digests, a gradient of 5-45% (v/v) acetonitrile in 0.1% formic acid (or TFA, if MALDI MS/MS is available) over 45 min, and then 45-95% acetonitrile in 0.1% formic acid (or TFA, if MALDI MS/MS is available) over 5 min can be used. 0.1% Formic acid solution is used on the Q-TOF instrument and 0.1% TFA solution is used on the Dionex Probot fraction collector for off-line coupling between HPLC and MALDI-MS/MS analysis (carried out on the ABI 4700). For a complex sample, a gradient of 5-45% (v/v) acetonitrile over 90 min, and then 45-95% acetonitrile over 30 min is used. For a very complex sample, a gradient of 5-45% (v/v) acetonitrile over 120 min, and then 45-95% acetonitrile over 60 min might be used. On the Q-TOF, one survey scan and four MS/MS data channels are used to acquire CID data with 1.4 s scan time. On the 4700 proteomics, the most intense eight peptides with mass over 1000 are chosen for MS/MS analysis.

Identification of Biomarkers

Software programs such as MSQuant can be used for quantification of protein expression (msquant.sourceforge.net).

Biomarkers are identified as proteins having an abundance in disease state cells that is either greater than or less than that in normal cells, or having an abundance in a given cellular compartment or fraction of disease state cells that is greater or less than that in the same cellular compartment or fraction of normal cells. The amount by which the abundance of a protein can differ between disease state and normal cells to be identified as a biomarker, for example, can be greater than 20%, greater than 30%, greater than 50%, greater than 70%, greater than 90%, or greater than 100%. For example, a biomarker can be an identified protein whose abundance in disease state cells is at least 200% (or 2-fold) greater than or less than its abundance in normal cells. In other cases, a biomarker can be an identified protein whose abundance in disease state cells is at least 300% (or 3-fold) greater than or less than its abundance in normal cells. In yet other cases, a biomarker can be an identified protein whose abundance in disease state cells is at least 500% (or 5-fold) greater than or less than its abundance in normal cells.

A protein identified as a biomarker can be a previously characterized protein or a protein that has not been previously characterized. Preferably, mass spectrometry analysis provides amino acid sequence of peptides of proteins that differ in abundance between normal and disease state cultures, and such amino acid sequences can be compared with nucleic acid and protein sequence databases. Where antibodies are unavailable for characterized or uncharacterized proteins, they can be generated using methods known in the art using synthetic peptides or recombinant or purified protein. Such antibodies can be used to validate biomarkers as well as for detection of biomarkers in tissues samples.

An advantage of using SILAC/MS to identify biomarkers of disease state cells is that the method provides an extensive, and, in principle, complete profile of the proteins expressed by cells that belong to the class of proteins targeted in the separation methods (for example, membrane proteins, phosphoproteins, nuclear proteins). Thus, using the methods of the present invention users can identify multiple biomarkers for a disease state. The identification of multiple biomarkers can allow for more reliable detection methods, where cells of a disease state can be identified, and potentially classified, by analysis of expression levels of multiple proteins.

Biomarkers can be validated by confirming expression differences between normal and disease state cells using methods of detecting protein levels other than SILAC. For example, protein level comparisons can be performed using immunocytochemistry, Western blot, immunoprecipitation, ELISA, or other antibody binding and detection methods.

The invention also includes antibodies to biomarkers identified using the methods disclosed herein, including the proteins listed in Table 1 and Table 2. Methods of generating antibodies to proteins are well known in the art. For example, polyclonal antibodies may be isolated and purified from vaccinated animals using procedures well-known in the art (for example, see Harlow et al., Antibodies: A Laboratory Manual, Cold Spring Harbor Laboratory Press, 1988).

Antibodies of the invention can also be monoclonal antibodies generated against proteins identified using the methods disclosed herein, such as the proteins listed in Table 1 and Table 2. For example, monoclonal antibodies can be produced following the procedure of Kohler and Milstein (Nature 256:495-497 (1975) (for example, see Harlow et al., supra). Briefly, monoclonal antibodies can be produced by immunizing mice with a biomarker protein, such as a protein of Table 1 or Table 2, verifying the presence of antibody production by removing a serum sample and testing for reactivity against the biomarker protein, removing the spleen to obtain B-lymphocytes, fusing the B-lymphocytes with myeloma cells to produce hybridomas, cloning the hybridomas, selecting positive clones which produce anti-biomarker antibody, culturing the anti-biomarker antibody-producing clones, and isolating anti-biomarker antibodies from the hybridoma cultures.

Antibodies of the invention can also be antibody fragments that specifically bind the proteins identified using the methods disclosed herein, such as the proteins listed in Table 1 and Table 2.

Antibodies of the invention also include engineered antibodies, including, without limitation, humanized antibodies, single-chain antibodies, and recombinant antibodies optimized through phage display. In some embodiments antibodies or antibody fragments can be isolated from antibody phage libraries generated, for example using the techniques described in McCafferty et al. (1990) Nature 348: 552-554, using the antigen of interest (such as a biomarker identified by the methods provided herein) to select for a suitable antibody fragment. Clackson et al. (1991) 352: 624-628 and Marks et al. (1991) J. Mol. Biol. 22: 581-597 describe the isolation of murine and human antibodies, respectively, using phage libraries. Subsequent publications describe the production of high affinity (nanomolar range) human antibodies by chain shuffling (Mark et al. (1992) Bio Technol. 10: 779-783), as well as combinatorial infection and in vivo recombination as a strategy for constructing very large phage libraries (Waterhouse et al. (1993) Nuc. Acids Res. 21: 2265-2266). The invention includes bacterial lines comprising phage libraries and phage clones of antibodies generated against biomarkers of the invention, such as the biomarkers listed in Table 1 and Table 2. The invention includes eukaryotic and bacterial lines comprising nucleic acid constructs encoding antibodies generated against biomarkers of the invention, such as the biomarkers listed in Table 1 and Table 2.

The invention encompasses antibodies that specifically bind the proteins of Table 1 and Table 2, and hybridoma cell lines, bacterial cell lines, and phage that produce antibodies that specifically bind the proteins of Table 1 and Table 2. The invention also encompasses nucleic acid constructs that encode antibodies that specifically bind proteins of Table 1 and Table 2.

In one aspect, the invention includes an antibody that specifically binds a protein of Table 2 that display a five-fold or greater difference in expression in breast cancer cells when compared with normal cells, such as annexin V chain c (SEQ ID NO:7; Genbank gi 809190), epididymal protein (SEQ ID NO:17; Genbank gi 23092553), glutaminase (SEQ ID NO:19; Genbank gi 12044394), inter-alpha-trypsin inhibitor heavy chain 3 precursor (SEQ ID NO:25; Genbank gi 3024064), alpha 2 macroglobulin (SEQ ID NO:29; Genbank gi 224053), membrane alanine aminopeptidase precursor (SEQ ID NO:30; Genbank gi 37590640; Genbank gi 4502095), type-2 phosphatidic acid phosphohydrolase (SEQ ID NO:33; Genbank gi 3015569), pregnancy zone protein precursor (SEQ ID NO:36; Genbank gi 131756), transglutaminase 2 isofomm a/transglutaminase C (SEQ ID NO:42; Genbank gi 39777597), the unnamed protein product having a sequence with NCBI database Genbank gi 7023123 (SEQ ID NO:47), autoantigen p542 (SEQ ID NO:55; Genbank gi 3334899), the 34 kD nucleolar sclerodema antigen (SEQ ID NO:71; Genbank gi 3399667), lysosomal proteinase cathepsin b (SEQ ID NO:74; Genbank gi 181178), osteoblast-specific factor 2 (SEQ ID NO:79; Genbank gi 46576887), SP—H antigen (SEQ ID NO:84; Genbank gi 743447), thyroid-lupus autoantigen p70 (SEQ ID NO:91; Genbank gi 4503841), the unnamed protein product having NCBI database Genbank gi 32097 (SEQ ID NO:96), the unnamed protein product having NCBI database Genbank gi 7022744 (SEQ ID NO:97), the unnamed protein product having NCBI database Genbank gi 21749696 (SEQ ID NO:98), a hypothetical protein (mago-nashi homolog) having NCBI database Genbank gi 15012020 (SEQ ID NO:107), a hypothetical protein (LS1-like) having NCBI database Genbank gi 15778927 (SEQ ID NO:108), a protein (human mRNA, complete cds gene product) having NCBI database Genbank gi 348239 (SEQ ID NO: 109), FTSJ3 protein (SEQ ID NO:110; Genbank gi 62914003), a predicted protein (similar to RIKEN cDNA 0610009D07) having NCBI having database Genbank gi 50745107(SEQ ID NO:111), chromosome segregation protein smc1 (SEQ ID NO:119; Genbank gi 2135244), CPSF6 protein (SEQ ID NO:123; Genbank gi 12653847), DNA-activated protein kinase (SEQ ID NO:167; Genbank gi 32140473) and DNA-activated protein kinase catalytic subunit (SEQ ID NO:126; Genbank gi 38258929), activating signal cointegrator 1 complex subunit 3-like 1 (SEQ ID NO:130; Genbank gi 40217847), heterogeneous nuclear ribonucleoprotein M, (SEQ ID NO:133; Genbank gi 55977747) a protein similar to small nuclear ribonucleoprotein D1 having NCBI database Genbank gi 34877889 (SEQ ID NO:142), histone deacetylase 2 (SEQ ID NO:144; Genbank gi 4557641), histone Hlb (SEQ ID NO:146; Genbank gi 356168), histone H2A.5 (SEQ ID NO:152; Genbank gi 70686), a protein similar to histone H3 having NCBI database Genbank gi 30156584 (SEQ ID NO:153), histone H3 (SEQ ID NO:158; Genbank gi 386772), histone H4 (SEQ ID NO:159; Genbank gi 223582), nucleolar protein NOP5/NOP58 (SEQ ID NO:107; Genbank gi 17380155; SEQ ID NO:164; Genbank gi 21595782), PTB-associated splicing factor (SEQ ID NO:168; Genbank gi 38458), or SmB/B′ autoimmune antigen (SEQ ID NO:180; Genbank gi 36495). In some preferred embodiments, breast cancer can be detected in a subject by detecting the expression level of one or more of the proteins of Table 2 that display a five-fold or greater difference in expression in breast cancer cells when compared with normal cells.

The antibodies of the invention can be used in detection of biomarkers, relative or absolute quantitation of biomarkers, diagnosis of a disease state (such as diagnosis of cancer), cancer typing, cancer staging, or prognosis. Detection of biomarkers can be by immunoassays of any type, which can be performed in solution phase (for example, ELISA protocols) or on a substrate, such as a membrane, slide, or bead. Immunoassays are well know in the art. Various methods of generating antibodies and methods for immunoassays are disclosed, for example, in U.S. Pat. No. 6,828,110; U.S. Pat. No. 6,828,110; U.S. Pat. No. 6,828,110; U.S. Pat. No. 6,218,109; U.S. Pat. No. 5,849,508; and U.S. Pat. No. 5,693,778; all herein incorporated by reference in their entireties. Biomarkers can also be validated by detecting differences in levels of nucleic acids that encode the biomarker proteins. Such detection can be, for example, by Northern blot, microarray hybridization, RT-PCR or other polymerase-based assays, CISH, or FISH. Biomarkers can be validated in the same cells used in the SILAC experiments used to identify the biomarkers, but preferably biomarkers are validated in a plurality of cell isolates, and preferably one or more of the cell isolates used to validate the biomarkers is different from the cell isolates used to identify the biomarkers using SILAC. Biomarkers identified in cancer cell fractions (including cytosolic, nuclear, or membrane fractions) using SILAC, as well as biomarkers found to be present in the cell media, can also validated by testing for their presence in patient sera, blood, plasma, lymphatic fluid aspirates or lavages.

Diagnosis of Disease Using Biomarkers

The abundance of one or more biomarkers in a biological sample can be correlated with a disease state, such as cancer. An altered expression pattern of one or more biomarkers in a biological sample can be correlated with the correlated with disease, such as cancer. The altered expression pattern can be, as nonlimiting examples, a different subcellular localization or post-translational modification.

One or more biomarkers of a disease state identified using the methods of the present invention can be detected in a plurality of biological samples in which cells of the tissue have been confirmed as having a particular disease state. Preferably, the abundance or subcellular localization of the biomarkers in disease tissue is evaluated at the same times as the abundance of the biomarkers in biological samples of non-disease tissue. Statistical analysis can be performed to determine correlates of the abundance of particular biomarkers with the disease state.

For example, as described in Example 1, the methods of the present invention have been used to identify breast cancer biomarkers. The example demonstrates the identification of biomarkers for cancer using cultures of normal and pathological cells grown in media that differs by the inclusion in one culture of heavy isotope-containing metabolic precursors and mass spectrometry to compare the relative abundance of large numbers of proteins. The abundance of such biomarkers in biological samples taken from breast cancer patients and sample of non-cancerous breast tissue can be determined using any reliable detection methods, including those disclosed herein.

Correlation of Biomarkers with Disease Type, Stage, Prognosis, and Response to Therapy

The abundance of one or more biomarkers in a biological sample can be correlated with a type, stage, or prognosis of a disease that the biomarker is indicative of. The abundance of a biomarker in a biological sample can also be correlated with response of the disease to particular therapies, such as drugs. One or more biomarkers of a disease state identified using the methods of the present invention can be detected in plurality of biological samples in which cells of the tissue have been confirmed as having a particular disease state, and have been classified according to one or more of disease type, disease stage, disease prognosis, or response of the patient to a given treatment. Preferably, the abundance of the biomarkers in disease tissue is evaluated at the same times as the abundance of the biomarkers in biological samples of non-disease tissue. Statistical analysis can be performed to determine correlates of the abundance of particular biomarkers with these parameters.

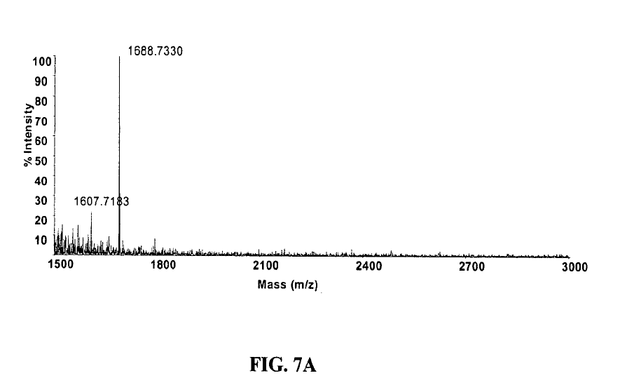

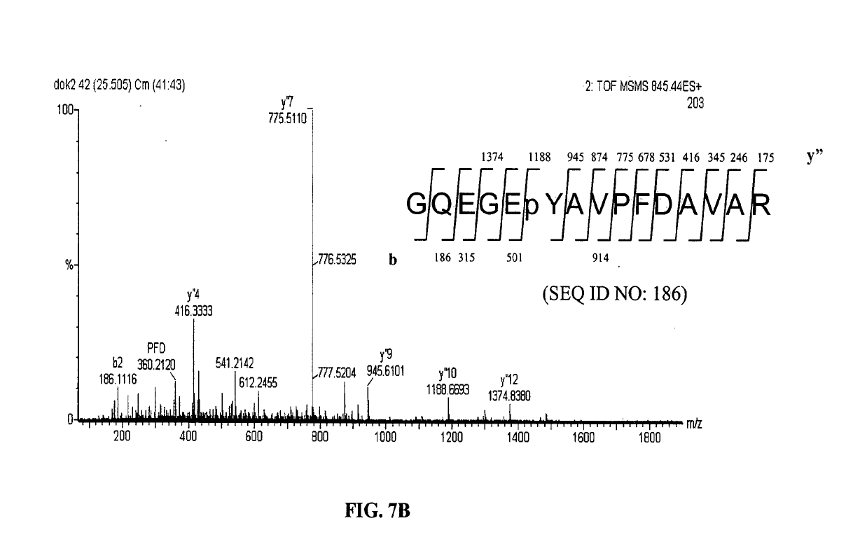

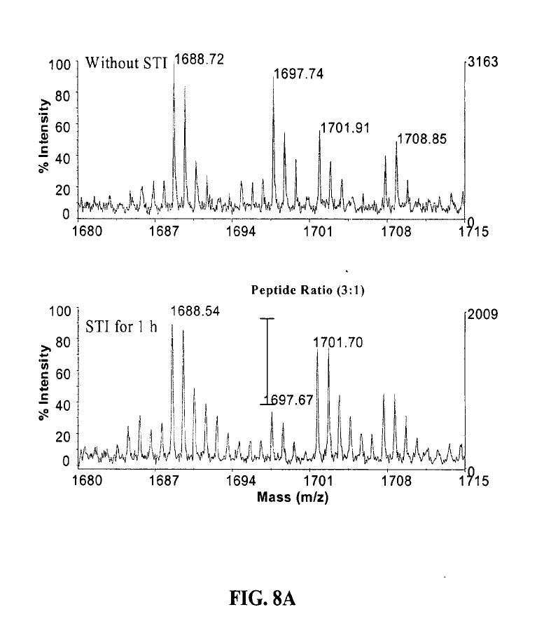

For example, as described in Example 1, the methods of the present invention have been used to identify breast cancer biomarkers. Seventy-five proteins were identified as having a three-fold or greater abundance in breast cancer cells than in normal breast cells. As depicted in FIG. 7 and Table 5, biomarkers osteoblast specific factor-2 (OSF-2) (SEQ ID NO:79, Genbank gi 46576887), DNA-activated protein kinase (SEQ ID NO:167, Genbank gi 32140473), membrane alanine aminopeptidase (CD13) (SEQ ID NO:30; Genbank gi 37590640) and alpha-2 macroglobulin (SEQ ID NO:29; Genbank gi 224053) tested by antibody staining of tissue or Western blot of normal breast tissue and breast cancer cells were found to have altered expression in breast cancer cells when compared with normal breast cells. Thus, the detection of biomarkers using SILAC is reliable and verifiable using other methods for comparison of expression levels or patterns.

The abundance of such biomarkers in biological samples taken from breast cancer patients can be determined using any reliable detection methods, including those disclosed herein. The National Cancer Institute maintains the Cooperative Breast Cancer Tissue Resource (www.cbctr.nci.nih.gov) to supply researchers with primary breast cancer tissues and associated clinical data. Analysis of expression of breast cancer biomarkers, such as those disclosed herein, can be examined in tissues of this tissue bank, for example, and correlated with pathological and clinical information that is available. Such correlates can be used for diagnosing, typing, and staging of cancer using biomarker detection on biological samples of patients known to have or suspected of having cancer. Such correlates can also be used for determining the probability of response of the patient to anticancer agents and a prognosis.

Biomarkers of a disease state identified using SILAC/MS can also be candidate drug targets. Proteins that are overexpressed in a disease state cell, such as a cancer cell, can also be used for drug targeting. This is particularly relevant to biomarkers for disease state cells identified after isolation of cell membranes. Immunohistochemistry demonstrates that OSF-2 is expressed on the cell membrane of cancer cells, whereas OSF-2 is not found on the cell membrane of normal cells, but rather can be detected in nuclei. The present invention allows identification of proteins that are expressed differentially in particular cellular compartments, such as the cell membrane. Proteins that are overexpressed in cell membranes of cancer cells with respect to normal cells, for example, can be used to develop affinity reagents for the cancer cell membrane proteins that can be conjugated to anticancer drugs.

In another application, biomarkers identified by SILAC/MS using stem cell cultures can be used to determine the differentiation state of cells, such as but not limited to stem cells used for therapeutic purposes.

### Illustrative Biomarkers

Illustrative biomarkers of present invention include those whose expression levels is discovered to differ by at least two-fold between breast cancer cells and normal breast cells. Such biomarkers are listed in Table 1. More preferred biomarkers that differ by at least three-fold between breast cancer cells and normal breast cells. Such biomarkers are listed in Table 2. Some exemplary biomarkers include breast cancer biomarkers osteoblast specific factor-2 (OSF-2) (SEQ ID NO:79, Genbank gi 46576887), DNA-activated protein kinase (SEQ ID NO:167, Genbank gi 32140473), membrane alanine aminopeptidase (CD13) (SEQ ID NO:30; Genbank gi 37590640) and alpha-2 (macroglobulin SEQ ID NO:29; Genbank gi 224053).

The proteins whose differential expression by breast cancer and normal breast cells was demonstrated by the methods of the invention were identified by database searching. Thus the names of the proteins may include “precursor” or “isoform” because these reflect the title of the sequence entries in the sequence database. The biomarkers of the invention are not limited to particular forms of these proteins, however, and encompass all fomms of a protein encoded at a particular locus by a particular gene that encodes a protein listed herein. A biomarker of the invention thus includes a protein listed in Table 1 or Table 2 and includes alternative isoforms, processed forms, and post-translationally modified forms of the listed proteins.

### II. Use of Biomarkers in Detecting Protein Expression in Disease State Cells

Biomarkers for a disease state identified by the methods disclosed herein can be used to detect expression of proteins in cells of a disease state, such as, but not limited to, cancer cells. For example, as outlined in Section I, above, disease state cells such as cancer cells and normal cells of the same type can be grown in parallel cultures, in which either the disease state cell culture or the normal cell culture contains one or more heavy isotope amino acids. After growth of cells in culture, such that essentially all of the protein in the heavy isotope culture is labeled, equal numbers of the cells can be mixed, and the cells can be subjected to cell fractionation, protein separation, and protein digestion. Peptides resulting from protein digestion of the pooled cell culture samples can be analyzed by mass spectrometry and, preferably, multiple biomarkers can be identified in which the biomarker is present at different level in disease state cells and normal cells.

In further experiments, one or more of the identified biomarkers can be detected in one or more biological test samples, such as tissue samples or bodily fluid samples. The sample can be a tumor biopsy sample, a blood, plasma, or serum sample, lymphatic fluid, saliva, a lung aspirate, a nipple aspirate, breast duct lavage sample, a pelvic lavage sample, a swab or scraping, etc. The sample need not be of the same tissue that was used to identify biomarkers using SILAC. For example, biomarkers that are overexpressed in cancer cells relative to normal cells that are localized within cancer cells or in or on the cell membranes of cancer cells may be detected in the blood or lymph, and thus blood, plasma, serum, and lymphatic fluid can be used to detect a disease state by detecting the presence of one or more biomarkers. Fluid samples used to harvest cells, cell fragments, and proteins at or near the site of a tumor (such as aspirates or ravages) can also be samples for detecting one or more biomarkers to diagnose a disease state. In some preferred embodiments, the biological test sample is a biological sample from a subject suspected of having breast cancer, for example, a serum sample, a breast tumor biopsy sample, a nipple aspirate, or a breast duct lavage sample.

The present invention also includes methods of detecting one or more biomarkers expressed by a cancer cell, where the biomarker is a protein of Table 1, or a nucleic acid encoding at least a portion of a protein of Table 1, comprising detecting in a biological sample of a patient known to have or suspected of having cancer, an expression level of a protein of Table 1, or a nucleic acid encoding the protein of Table 1, wherein an altered expression level in the biological sample compared to the expression level in a normal sample is indicative of a cancerous state. Biomarkers identified by the methods of the present invention are not limited to proteins having the database sequences of the identified biomarker protein, but also include proteins encoded by the same gene at the same chromosomal locus as the identified biomarker. Thus, detecting a biomarker of Table 1 also includes detecting a protein encoded by allelic variants of those identified as encoding the proteins listed in Table 1, or protein variants resulting from one or more mutations or from alternative splicing of the encoding gene, or proteins differing from the proteins listed in Table 1 in post-translational modifications, including but not limited to proteolytic processing.

Data provided herein identifies the proteins of Table 1 as being proteins that are differentially expressed in human breast cancer cells. Accordingly, provided herein is a method of detecting one or more biomolecules, comprising detecting in a biological sample, expression of a protein of Table 1, or a nucleic acid encoding a protein of Table 1, wherein said biological sample is a sample of a patient with a breast pathology. The biological sample can be, for example, a tumor biopsy sample or a breast tumor biopsy sample. Furthermore, the sample can be a fluid sample. For example, sample of blood, plasma, serum, urine, saliva, lymphatic fluid, pelvic lavage, lung aspirate, nipple aspirate, or breast duct lavage.

In certain aspects of the invention, expression is detected of two or more, three or more, four or more, five or more, six or more, seven of more, eight or more, nine or more, ten or more, twenty-five or more, one-half, one-third, or all of the proteins of Table 1, or of nucleic acids encoding two or more, three or more, four or more, five or more, six or more, seven of more, eight or more, nine or more, ten or more, twenty-five or more, one-half, one-third, or all of the proteins of Table 1.

In some illustrative aspects, expression is detected for one or more proteins of Table 2, or nucleic acids encoding one or more proteins of Table 2. Furthermore, in certain aspects of the invention, expression is detected of two or more, three or more, four or more, five or more, six or more, seven of more, eight or more, nine or more, ten or more, twenty-five or more, one-half, one-third, or all of the proteins of Table 2, or of nucleic acids encoding two or more, three or more, four or more, five or more, six or more, seven of more, eight or more, nine or more, ten or more, twenty-five or more, one-half, one-third, or all of the proteins of Table 2.

Furthermore, expression can be detected for one or more, two or more, three or more, four or more, five or more, six or more, seven of more, eight or more, nine or more, ten or more, twenty-five or more, one-half, one-third, or all of the following proteins, or nucleic acids encoding the protein(s): annexin V chain c (SEQ ID NO:7), epididymal protein (SEQ ID NO:17), glutaminase (SEQ ID NO:19), inter-alpha-trypsin inhibitor heavy chain 3 precursor (SEQ ID NO:25), alpha 2 macroglobulin (SEQ ID NO:29), membrane alanine aminopeptidase precursor (SEQ ID NO:30), type-2 phosphatidic acid phosphohydrolase (SEQ ID NO:33), pregnancy zone protein precursor (SEQ ID NO:36), transglutaminase 2 isoform a/transglutaminase C (SEQ ID NO:42), the unnamed protein product having a sequence with NCBI database Genbank gi 7023123 (SEQ ID NO:47), autoantigen p542 (SEQ ID NO:55), the 34 kD nucleolar sclerodema antigen (SEQ ID NO:71), lysosomal proteinase cathepsin b (SEQ ID NO:74), osteoblast-specific factor 2 (SEQ ID NO:79), SP—H antigen (SEQ ID NO:84), thyroid-lupus autoantigen p70 (SEQ ID NO:91), the unnamed protein product having NCBI database Genbank gi 32097 (SEQ ID NO:96), the unnamed protein product having NCBI database Genbank gi 7022744 (SEQ ID NO:97), the unnamed protein product having NCBI database Genbank gi 21749696 (SEQ ID NO:98), a hypothetical protein (mago-nashi homolog) having NCBI database Genbank gi 15012020 (SEQ ID NO:107), a hypothetical protein (LS 1-like) having NCBI database Genbank gi 15778927 (SEQ ID NO:108), a protein (human mRNA, complete cds gene product) having NCBI database Genbank gi 348239 (SEQ ID NO:109), FTSJ3 protein (SEQ ID NO:110), a predicted protein (similar to RIKEN cDNA 0610009D07) having NCBI having database Genbank gi 50745107(SEQ ID NO:111), chromosome segregation protein smc1 (SEQ ID NO:119), CPSF6 protein (SEQ ID NO:123), DNA-activated protein kinase (SEQ ID NO:167) and DNA-activated protein kinase catalytic subunit (SEQ ID NO:126), activating signal cointegrator I complex subunit 3-like 1 (SEQ ID NO:130), heterogeneous nuclear ribonucleoprotein M, (SEQ ID NO:133) a protein similar to small nuclear ribonucleoprotein D1 having NCBI database Genbank gi 34877889 (SEQ ID NO:142), histone deacetylase 2 (SEQ ID NO:144), histone H1b (SEQ ID NO:146), histone H2A.5 (SEQ ID NO:152), a protein similar to histone H3 having NCBI database Genbank gi 30156584 (SEQ ID NO:153), histone H3 (SEQ ID NO:158), histone H4 (SEQ ID NO:159), nucleolar protein NOP5/NOP58 (SEQ ID NO:107; SEQ ID NO:164), PTB-associated splicing factor (SEQ ID NO:168), and SmB/B′ autoimmune antigen (SEQ ID NO:180).

Furthermore, expression can be detected for one or more, two or more, three or more, four or more, five or more, six or more, seven of more, eight or more, nine or more, ten or more, twenty-five or more, one-half, one-third, or all of the following proteins, or nucleic acids encoding the protein(s): annexin V chain c (SEQ ID NO:7), epididymal protein (SEQ ID NO:17), glutaminase (SEQ ID NO:19), inter-alpha-trypsin inhibitor heavy chain 3 precursor (SEQ ID NO:30), alpha 2 macroglobulin (SEQ ID NO:29), membrane alanine aminopeptidase precursor (SEQ ID NO:31), type-2 phosphatidic acid phosphohydrolase (SEQ ID NO:33), pregnancy zone protein precursor (SEQ ID NO:36), transglutaminase 2 isoform a/transglutaminase C (SEQ ID NO:42), and the unnamed protein product having a sequence with NCBI database Genbank gi 7023123 (SEQ ID NO:47).

Furthermore, expression can be detected for one or more, two or more, three or more, four or more, five or more, six or more, seven of more, eight or more, nine or more, ten or more, twenty-five or more, one-half, one-third, or all of the proteins, or encoding nucleic acids of: autoantigen p542 (SEQ ID NO:55), the 34 kD nucleolar sclerodema antigen (SEQ ID NO:71), lysosomal proteinase cathepsin b (SEQ ID NO:74), osteoblast-specific factor 2 (SEQ ID NO: 79), SP—H antigen (SEQ ID NO:84), thyroid-lupus autoantigen p70 (SEQ ID NO:9), the unnamed protein product having NCBI database Genbank gi 32097 (SEQ ID NO:96), the unnamed protein product having NCBI database Genbank gi 7022744 (SEQ ID NO:97), the unnamed protein product having NCBI database Genbank gi 21749696 (SEQ ID NO:98), a hypothetical protein (mago-nashi homolog) having NCBI database Genbank gi 15012020 (SEQ ID NO:107), a hypothetical protein (LS1-like) having NCBI database Genbank gi 15778927 (SEQ ID NO:108), a protein (human mRNA, complete cds gene product) having NCBI database Genbank gi 348239 (SEQ ID NO:109), FTSJ3 protein (SEQ ID NO:110), a predicted protein (similar to RIKEN cDNA 0610009D07) having NCBI having database Genbank gi 50745107 (SEQ ID NO:11l), chromosome segregation protein smc1 (SEQ ID NO:119; Genbank gi 2135244), CPSF6 protein (SEQ ID NO:123), DNA-activated protein kinase (SEQ ID NO:167) and DNA-activated protein kinase catalytic subunit (SEQ ID NO:126), activating signal cointegrator 1 complex subunit 3-like 1 (SEQ ID NO:130), heterogeneous nuclear ribonucleoprotein M (SEQ ID NO:133), a protein similar to small nuclear ribonucleoprotein D1 having NCBI database Genbank gi 34877889 (SEQ ID NO:142), histone deacetylase 2 (SEQ ID NO:144), histone H1b ((SEQ ID NO:146), histone H2A.5 (SEQ ID NO:152), a protein similar to histone H3 having NCBI database Genbank gi 30156584 (SEQ ID NO:153), histone H3 (SEQ ID NO:158), histone H4 (SEQ ID NO:159), nucleolar protein NOP5/NOP58 (SEQ ID NO:162), PTB-associated splicing factor (SEQ ID NO:168), or SmB/B′ autoimmune antigen (SEQ ID NO:180).

Certain aspects of the invention provide quantitative methods. For example, in certain aspects an expression level is determined of a protein of Table 1, or a nucleic acid encoding a protein of Table 1. In certain embodiments, an altered expression level in the biological sample compared to a normal sample is indicative of the presence of a breast pathology, such as breast cancer. Furthermore, expression levels can be correlated with a type of cancer, a stage of cancer, a prognosis, and/or response to one or more anti-cancer agents.

Typically, methods of this embodiment of the invention are performed by contacting the biological sample with a specific binding reagent that binds to the protein or the nucleic acid. Expression levels can then be quantitated by measuring the amount of specific binding reagent that binds to biomolecules in the sample. The specific binding reagent is typically an antibody or a nucleic acid. In certain examples, the antibody can bind a secondary modification of a protein of Table 1 or Table 2. It will be recognized that the detection method can be an immunoassay.

In another embodiment, provided herein is a kit that includes a specific binding reagent that binds to a protein of Table 1, or that binds to a nucleic acid encoding a protein of Table 1. Furthermore, the kits typically include a control that is a biological sample derived from a subject having a breast pathology. For example, the control can include cells obtained directly from a subject having a breast pathology, or tissue culture cells derived from cells of a subject having a breast pathology, such as breast cancer or a breast tumor The specific binding reagent of the kit is typically an antibody or a nucleic acid. The specific binding reagent is typically present in one or more tubes that are associated together in packaging and shipped from a manufacturer to a customer.

The kit can include additional specific binding reagents. For example, specific binding reagents that bind to one or more, two or more, three or more, four or more, five or more, six or more, seven of more, eight or more, nine or more, ten or more, twenty-five or more, one-half, one-third, or all of the proteins, or encoding nucleic acids of Table 1.

In one embodiment, the kit includes specific binding reagents that bind to one or more, two or more, three or more, four or more, five or more, six or more, seven of more, eight or more, nine or more, ten or more, twenty-five or more, one-half, one-third, or all of the proteins, or encoding nucleic acids of Table 2.

In one embodiment, the kit includes specific binding reagents that bind to one or more, two or more, three or more, four or more, five or more, six or more, seven of more, eight or more, nine or more, ten or more, twenty-five or more, one-half, one-third, or all of the following proteins, or nucleic acids encoding these proteins: annexin V chain c (SEQ ID NO:7), epididymal protein (SEQ ID NO:17), glutaminase (SEQ ID NO:19), inter-alpha-trypsin inhibitor heavy chain 3 precursor (SEQ ID NO:25), alpha 2 macroglobulin (SEQ ID NO:29), membrane alanine aminopeptidase precursor (SEQ ID NO:30), type-2 phosphatidic acid phosphohydrolase (SEQ ID NO:33), pregnancy zone protein precursor (SEQ ID NO:36), transglutaminase 2 isoform a/transglutaminase C (SEQ ID NO:42), the unnamed protein product having a sequence with NCBI database Genbank gi 7023123 (SEQ ID NO:47), autoantigen p542 (SEQ ID NO:55), the 34 kD nucleolar sclerodema antigen (SEQ ID NO:71), lysosomal proteinase cathepsin b (SEQ ID NO:74), osteoblast-specific factor 2 (SEQ ID NO:79), SP—H antigen (SEQ ID NO:84), thyroid-lupus autoantigen p70 (SEQ ID NO:91), the unnamed protein product having NCBI database Genbank gi 32097 (SEQ ID NO:96), the unnamed protein product having NCBI database Genbank gi 7022744 (SEQ ID NO:97), the unnamed protein product having NCBI database Genbank gi 21749696 (SEQ ID NO:98), a hypothetical protein (mago-nashi homolog) having NCBI database Genbank gi 15012020 (SEQ ID NO:107), a hypothetical protein (LS1-like) having NCBI database Genbank gi 15778927 (SEQ ID NO:108), a protein (human mRNA, complete cds gene product) having NCBI database Genbank gi 348239 (SEQ ID NO:109), FTSJ3 protein (SEQ ID NO:110), a predicted protein (similar to RIKEN cDNA 0610009D07) having NCBI having database Genbank gi 50745107(SEQ ID NO:11l), chromosome segregation protein smc1 (SEQ ID NO:119), CPSF6 protein (SEQ ID NO:123), DNA-activated protein kinase (SEQ ID NO:167) and DNA-activated protein kinase catalytic subunit (SEQ ID NO:126), activating signal cointegrator 1 complex subunit 3-like 1 (SEQ ID NO:130), heterogeneous nuclear ribonucleoprotein M, (SEQ ID NO:133) a protein similar to small nuclear ribonucleoprotein D1 having NCBI database Genbank gi 34877889 (SEQ ID NO:142), histone deacetylase 2 (SEQ ID NO:144), histone Hlb (SEQ ID NO:146), histone H2A.5 (SEQ ID NO:152), a protein similar to histone H3 having NCBI database Genbank gi 30156584 (SEQ ID NO:153), histone H3 (SEQ ID NO:158), histone H4 (SEQ ID NO:159), nucleolar protein NOP5/NOP58 (SEQ ID NO:107; SEQ ID NO:164), PTB-associated splicing factor (SEQ ID NO:168), or SmB/B′ autoimmune antigen (SEQ ID NO:180).

In one embodiment, the kit includes specific binding reagents that bind to one or more, two or more, three or more, four or more, five or more, six or more, seven of more, eight or more, nine or more, ten or more, twenty-five or more, one-half, one-third, or all of the following proteins, or nucleic acids encoding these proteins: annexin V chain c (SEQ ID NO:7), epididymal protein (SEQ ID NO:17), glutaminase (SEQ ID NO:19), inter-alpha-trypsin inhibitor heavy chain 3 precursor (SEQ ID NO:30), alpha 2 macroglobulin (SEQ ID NO:29), membrane alanine aminopeptidase precursor (SEQ ID NO:31), type-2 phosphatidic acid phosphohydrolase (SEQ ID NO:33), pregnancy zone protein precursor (SEQ ID NO:36), transglutaminase 2 isoform a/transglutaminase C (SEQ ID NO:42), or the unnamed protein product having a sequence with NCBI database Genbank gi 7023123 (SEQ ID NO:47).

In some preferred embodiments, one or more of the proteins listed in Table 1, or one or more nucleic acids encoding one or more of the proteins of Table 1, is detected in a biological sample of a patient known to have or suspected of having breast cancer, in which an altered expression level in the biological sample of the patient compared to the expression level in a normal sample is indicative of breast cancer. In some preferred embodiments, one or more of the proteins listed in Table 1, or one or more nucleic acids encoding one or more of the proteins of Table 1, is detected in a biological sample of a patient known to have or suspected of having breast cancer, in which an altered expression pattern biological sample of the patient compared to the expression pattern in a normal sample is indicative of breast cancer. The altered expression pattern can be, as nonlimiting examples, a different abundance, subcellular localization, aggregation status, activity, or post-translational modification. Detection of one or more biomarkers to detect breast cancer can be detection of two or more biomarkers detection of three or more biomarkers, or detection of four or more biomarkers of Table 1.

The present invention also includes methods of detecting one or more biomarkers expressed by a cancer cell, where the biomarker is a protein of Table 2, or a nucleic acid encoding at least a portion of a protein of Table 2, comprising detecting in a biological sample of a patient know to have or suspected of having cancer, an expression level or pattern of a protein of Table 2 or a nucleic acid encoding the protein of Table 2, in which an altered expression level or pattern in the biological sample compared to the expression level or pattern in a normal sample is indicative of a cancerous state. In some preferred embodiments, one or more of the proteins listed in Table 2 or one or more nucleic acids encoding one or more of the proteins of Table 2 is detected in a biological sample of a patient known to have or suspected of having breast cancer, wherein an altered expression level or pattern in the biological sample compared to the expression level in a normal sample is indicative of breast cancer. An altered expression pattern can be, as nonlimiting examples, a different abundance, subcellular localization, aggregation status, activity, or post-translational modification. Detection of one or more biomarkers to detect breast cancer can be detection of two or more biomarkers detection of three or more biomarkers, or detection of four or more biomarkers of Table 2.

Detecting a biomarker of Table 2 also includes detecting a protein encoded at the same genetic locus as those identified for the biomarkers of Table 2 (and as listed in Table 3), including proteins encoded by allelic variants of the genes identified as encoding the proteins listed in Table 2, or protein variants resulting from mutations or from alternative splicing, or proteins differing from the proteins listed in Table 2 in post-translational modifications, including but not limited to proteolytic processing.

In some preferred embodiments, cancer can be detected in a subject by detecting the expression level of one or more of the proteins of Table 2 that display a five-fold or greater difference in expression in breast cancer cells when compared with normal cells, such as annexin V chain c (SEQ ID NO:7; Genbank gi 809190), epididymal protein (SEQ ID NO:17; Genbank gi 23092553), glutaminase (SEQ ID NO:19; Genbank gi 12044394), inter-alpha-trypsin inhibitor heavy chain 3 precursor (SEQ ID NO:25; Genbank gi 3024064), alpha 2 macroglobulin (SEQ ID NO:29; Genbank gi 224053), membrane alanine aminopeptidase precursor (SEQ ID NO:30; Genbank gi 37590640; Genbank gi 4502095), type-2 phosphatidic acid phosphohydrolase (SEQ ID NO:33; Genbank gi 3015569), pregnancy zone protein precursor (SEQ ID NO:36; Genbank gi 131756), transglutaminase 2 isoform a/transglutaminase C (SEQ ID NO:42; Genbank gi 39777597), the unnamed protein product having a sequence with NCBI database Genbank gi 7023123 (SEQ ID NO:47), autoantigen p542 (SEQ ID NO:55; Genbank gi 3334899), the 34 kD nucleolar sclerodema antigen (SEQ ID NO:71; Genbank gi 3399667), lysosomal proteinase cathepsin b (SEQ ID NO:74; Genbank gi 181178), osteoblast-specific factor 2 (SEQ ID NO:79; Genbank gi 46576887), SP—H antigen (SEQ ID NO:84; Genbank gi 743447), thyroid-lupus autoantigen p70 (SEQ ID NO:91; Genbank gi 4503841), the unnamed protein product having NCBI database Genbank gi 32097 (SEQ ID NO:96), the unnamed protein product having NCBI database Genbank gi 7022744 (SEQ ID NO:97), the unnamed protein product having NCBI database Genbank gi 21749696 (SEQ ID NO:98), a hypothetical protein (mago-nashi homolog) having NCBI database Genbank gi 15012020 (SEQ ID NO:107), a hypothetical protein (LS1-like) having NCBI database Genbank gi 15778927 (SEQ ID NO:108), a protein (human mRNA, complete cds gene product) having NCBI database Genbank gi 348239 (SEQ ID NO:109), FTSJ3 protein (SEQ ID NO:110; Genbank gi 62914003), a predicted protein (similar to RIKEN cDNA 0610009D07) having NCBI having database Genbank gi 50745107(SEQ ID NO:111), chromosome segregation protein smc1 (Genbank gi 2135244), CPSF6 protein (Genbank gi 12653847), DNA-activated protein kinase (SEQ ID NO:119; Genbank gi 32140473) and DNA-activated protein kinase catalytic subunit (SEQ ID NO:126; Genbank gi 38258929), activating signal cointegrator 1 complex subunit 3-like 1 (SEQ ID NO:130; Genbank gi 40217847), heterogeneous nuclear ribonucleoprotein M, (SEQ ID NO:133; Genbank gi 55977747) a protein similar to small nuclear ribonucleoprotein D1 having NCBI database Genbank gi 34877889 (SEQ ID NO:142), histone deacetylase 2 (SEQ ID NO:144; Genbank gi 4557641), histone Hlb (SEQ ID NO:146; Genbank gi 356168), histone H2A.5 (SEQ ID NO:152; Genbank gi 70686), a protein similar to histone H3 having NCBI database Genbank gi 30156584 (SEQ ID NO:153), histone H3 (SEQ ID NO:158; Genbank gi 386772), histone H4 (SEQ ID NO:159; Genbank gi 223582), nucleolar protein NOP5/NOP58 (SEQ ID NO:107; Genbank gi 17380155; SEQ ID NO:164; Genbank gi 21595782), PTB-associated splicing factor (SEQ ID NO:168; Genbank gi 38458), or SmB/B′ autoimmune antigen (SEQ ID NO:180; Genbank gi 36495). In some preferred embodiments, breast cancer can be detected in a subject by detecting the expression level of one or more of the proteins of Table 2 that display a five-fold or greater difference in expression in breast cancer cells when compared with normal cells.

In some preferred embodiments, one or more of the proteins listed in Table 2 that display a five-fold or greater difference in expression in breast cancer cells when compared with normal cells is detected as having an altered expression pattern in the biological sample of the patient compared to the expression level in a normal sample. The altered expression pattern can be, as nonlimiting examples, a different abundance, subcellular localization, aggregation status, activity, or post-translational modification. Detection of an altered expression pattern of one or more biomarkers of Table 2 that display a five-fold or greater difference in expression in breast cancer cells when compared with normal cells to detect cancer can be detection of two or more biomarkers, detection of three or more biomarkers, or detection of four or more biomarkers of Table 2 that display a five-fold or greater difference in expression.

Detection methods can include detecting expression of one or more proteins of the proteins of Table 2 that display an increase in expression in breast cancer cells with respect to normal cells, or an increase in expression in a subcellular compartment of breast cancer cells when compared with expression of the protein in the subcellular compartment of normal cells, or an increased abundance in a biological sample from a patient known to have or suspected of having cancer when compared with a sample from a patient that does not have cancer.

Detection methods can include detecting expression of one or more proteins of the proteins of Table 2 that display a five-fold or greater increase in expression in breast cancer cells when compared with normal cells, such as autoantigen p542 (SEQ ID NO:55), the 34 kD nucleolar sclerodema antigen (SEQ ID NO:71), lysosomal proteinase cathepsin b (SEQ ID NO:74), osteoblast-specific factor 2 (SEQ ID NO:79), SP—H antigen (SEQ ID NO:84), thyroid-lupus autoantigen p70 (SEQ ID NO:91), the unnamed protein product having NCBI database Genbank gi 32097 (SEQ ID NO:96), the unnamed protein product having NCBI database Genbank gi 7022744 (SEQ ID NO:97), the unnamed protein product having NCBI database Genbank gi 21749696 (SEQ ID NO:98), a hypothetical protein having NCBI database Genbank gi 15012020 (SEQ ID NO:107), a hypothetical protein having NCBI database Genbank gi 15778927 (SEQ ID NO:108), a protein having NCBI database Genbank gi 348239 (SEQ ID NO:109), FTSJ3 protein (SEQ ID NO:110), a protein NCBI database Genbank gi 50745107 (SEQ ID NO:111), chromosome segregation protein smc1 (SEQ ID NO:119), CPSF6 protein (SEQ ID NO:123), DNA-activated protein kinase (SEQ ID NO:167), U5 snRNP Sm D1(Sm-D autoantigen) (SEQ ID NO:142), histone deacetylase 2 (SEQ ID NO:144), histone H1b (SEQ ID NO:146), histone H2A.5 (SEQ ID NO:152), histone H3, a protein similar to histone H3 (SEQ ID NO:158), having NCBI database Genbank gi 30156584 (SEQ ID NO:153), histone H4 (SEQ ID NO: 159), nucleolar protein NOP5/NO P58 (SEQ ID NO:107), PTB-associated splicing factor (SEQ ID NO:168), or SmB/B′ autoimmune antigen (SEQ ID NO:168).

In some preferred embodiments, cancer can be detected in a subject by detecting the expression level or expression pattern of one or more of osteoblast specific factor 2 (SEQ ID NO:79; OSF-2, periostin), membrane alanine aminopeptidase precursor (SEQ ID NO:30; CD13), DNA-activated protein kinase (SEQ ID NO:119), or alpha 2 macroglobulin (SEQ ID NO:29) in a biological sample of a patient known to have or suspected of having cancer. In some preferred embodiments, breast cancer can be detected in a subject by detecting the expression level or pattern of one or more of osteoblast specific factor 2 (SEQ ID NO:79; OSF-2, periostin), or membrane alanine aminopeptidase precursor (SEQ ID NO:30; CD13). As described in Example 1, using the methods of the present invention, these proteins were found to have different abundances in membranes isolated from breast cancer cells when compared with normal cells.

A biological test sample, or a fraction or extract thereof, can be tested for the presence, absence, or amount of one or more biomarkers, where the presence, absence, or amount of one or more biomarkers detected is indicative of the presence of cancer in the patient. The detection of a biomarker can use a biomarker binding reagent that specifically binds a biomarker, such as, for example, an antibody, or can use a biomarker binding reagent such as a nucleic acid molecule or nucleic acid analog that can specifically bind at least a portion of a nucleic acid that encodes a biomarker. Detection of the biomarker is by detection of a label that can be directly or indirectly bound to or can directly or indirectly bind a biomarker binding reagent. A biomarker binding reagent can comprise or be directly or indirectly bound to detectable labels as they are known in the art, including but not limited to, radioactive, fluorescent, luminescent, or colorimetric labels. It is also possible to directly or indirectly bind a signal generating molecule or system to a specific binding reagent that binds a biomarker. For example, enzymes such as, but not limited to luciferase can be directly or indirectly bound to a specific binding reagent. A detection step can optionally include the addition of further reagents, such as substrates or cofactors, that are required for signal generation. Biomarker detection reagents can also be designed such that they can be specifically bound by a labeled reagent during the detection procedure, as in “sandwich” hybridization. Nonlimiting examples of detection methods useful in the present invention include immunocytochemistry, Western blotting, ELISA, immunoprecipitation, protein array detection, and other methods that use specific binding reagents such as but not limited to antibodies, and methods that employ nucleic acid hybridization (such as, but not limited to, Northern blots, array hybridization, FISH) and polymerase based methods such as, but not limited to, RT-PCR.

Disease state cells or biomarkers derived from disease state cells can be identified using immunocytochemistry using an antibody that specifically binds the biomarker. In other preferred embodiments, the biomarker can be used to detect cells by immunoprecipitation, ELISA, or Western blot of cells, sample fluid, cell supernatants, or lysates prepared from the tissue sample.

In some preferred embodiments, a biomarker can be detected by detecting the nucleic acid that encodes the biomarker. Nucleic acid hybridization can be used, for example, Northern blot, array hybridization, or FISH can detect and, preferably, quantify nucleic acids encoding one or more biomarkers of a disease state.

One or more concentration steps, separation steps, or purification steps can optionally be performed on a biological sample prior to biomarker detection using the sample. For example, cells can be pelleted from fluid samples, and the cells can be further analyzed, or, alternatively, the supernatant of a centrifuged fluid sample can be analyzed for the presence or amount of one or more biomarkers. Where immunocytochemistry or FISH is used to detect disease state biomarkers, the cells can be fixed and prepared for antibody or nucleic acid binding using methods known in the art.

Cells obtained from tissue samples or fluid samples can optionally be lysed, and optionally, further fractionated or processed for protein or nucleic acid detection, for example, using immunoprecipitation, ELISA, or Western blot, or using nucleic acid hybridization or incorporation of nucleotides into nucleic acid molecules that encode at least a portion of a biomarker.

Preferably but optionally, detection of one or more biomarkers is quantitative or at least somewhat quantitative. By “somewhat quantitative” is meant that absolute amounts of the biomarker may not be determined, but amounts of biomarkers are determined relative to a standard, such as, for example, a standard of signal intensity based on comparison with controls that can be, for example, samples of normal cells, tissues, or biological samples, or fractions thereof. For example, the intensity of sample cell staining using a biomarker detection reagent can be scaled to the intensity of staining of one or more control cells. In another example, the amount of detection reagent bound to components isolated from biological test samples can also be compared with the amount of detection reagent bound to control components to calibrate levels of one or more biomarkers in the test sample.

The detection of the presence, absence, amount, or expression pattern of one or more biomarkers in a tissue sample or sample of bodily fluid can be indicative of a disease state. The sample can be a tumor biopsy sample, a blood, plasma, or serum sample, lymphatic fluid, saliva, a lung aspirate, a nipple aspirate, breast duct lavage sample, a pelvic lavage sample, a swab or scraping, etc. taken from a patient suspected of having or known to have cancer. The sample, or a fraction or extract thereof, can be tested for the presence, absence, or amount of one or more biomarkers, where the presence, absence, or amount of one or more biomarkers detected is indicative of the presence of cancer in the patient.

The detection of the presence, absence, amount, or expression pattern of one or more biomarkers in a tissue sample or sample of bodily fluid can be indicative of a type or stage of a disease. The sample can be a tumor biopsy sample, a blood, plasma, or serum sample, lymphatic fluid, saliva, a lung aspirate, a nipple aspirate, breast duct lavage sample, a pelvic lavage sample, a swab or scraping, etc. taken from a patient suspected of having or known to have cancer. The sample, or a fraction or extract thereof, can be tested for the presence, absence, or amount of one or more biomarkers, where the presence, absence, or amount of one or more biomarkers has been correlated with a type or stage of cancer. In this case, the presence or amount of one or more biomarkers detected in a patient sample can determine the type or stage of cancer in the patient. For example, the detection of the expression level of one or more proteins of Table 1 in a biological sample of a patient with cancer can be indicative of a type or stage of cancer, such as, but not limited to breast cancer. The detection of the expression level of one or more proteins of Table 2 can be indicative of a type or stage of cancer, such as, but not limited to breast cancer.

The detection of the presence, absence, amount, or expression pattern of one or more biomarkers in a tissue sample or sample of bodily fluid can be indicative of a prognosis of a disease, or the response of a disease to particular therapies. The sample can be a tumor biopsy sample, a blood, plasma, or serum sample, lymphatic fluid, saliva, a lung aspirate, a nipple aspirate, breast duct lavage sample, a pelvic lavage sample, a swab or scraping, etc. taken from a patient suspected of having or known to have cancer. The sample, or a fraction or extract thereof, can be tested for the presence, absence, amount, or expression pattern of one or more biomarkers, where the presence, absence, or amount of one or more biomarkers has been correlated with a prognosis or a response to anti-cancer agents of the cancer. In this case, the presence or amount of one or more biomarkers detected in a patient sample can determine a prognosis or predict a drug response of the patient. For example, the detection of the expression pattern of one or more proteins of Table 1 in a biological sample of a patient known to have or suspected of having cancer can be indicative of a prognosis or of cancer, such as, but not limited to breast cancer. The detection of the expression pattern of one or more proteins of Table 2 can be indicative of a prognosis of cancer, such as, but not limited to breast cancer.

In other embodiments, detection of the expression pattern of one or more proteins of Table 1 in a biological sample of a patient with cancer, such as, but not limited to breast cancer, can be used to predict a response to anti-cancer agents. In further embodiments, the detection of the expression level of one or more proteins of Table 2 can be used to predict a response of a patient with cancer, such as but not limited to breast cancer, to one or more anti-cancer agents.

Detection of biomarker to detect cancer, as well as detection of biomarkers for typing and staging of cancer, as well as detection of biomarkers to indicate prognosis of a disease, or the response of a disease to particular therapies, can be by detection of one or more biomarkers underexpressed by cancer cells with respect to normal cells, by detection of one or more biomarkers overexpressed by cancer cells with respect to normal cells, or by a combination of the two. For example, detection of biomarkers can be detection of the proteins of Table 2 identified as upregulated proteins. In addition or in the alternative, detection of biomarkers can be detection of the proteins of Table 2 identified as downregulated proteins.

Detection of biomarkers can be, for example detection of the proteins of Table 2 identified as upregulated proteins that are overexpressed by 5 fold or greater in cancer cells, such as one or more of autoantigen p542 (SEQ ID NO:55; Genbank gi 3334899), the 34 kD nucleolar sclerodema antigen (SEQ ID NO:71; Genbank gi 3399667), lysosomal proteinase cathepsin b (SEQ ID NO:74; Genbank gi 181178), osteoblast-specific factor 2 (SEQ ID NO: 79; Genbank gi 46576887), SP—H antigen (SEQ ID NO:84; Genbank gi 743447), thyroid-lupus autoantigen p70 (SEQ ID NO:9; Genbank gi 4503841), the unnamed protein product having NCBI database Genbank gi 32097 (SEQ ID NO:96), the unnamed protein product having NCBI database Genbank gi 7022744 (SEQ ID NO:97), the unnamed protein product having NCBI database Genbank gi 21749696 (SEQ ID NO:98), a hypothetical protein (mago-nashi homolog) having NCBI database Genbank gi 15012020 (SEQ ID NO:107), a hypothetical protein (LS1-like) having NCBI database Genbank gi 15778927 (SEQ ID NO:108), a protein (human mRNA, complete cds gene product) having NCBI database Genbank gi 348239 (SEQ ID NO:109), FTSJ3 protein (SEQ ID NO:110; Genbank gi 62914003), a predicted protein (similar to RIKEN cDNA 0610009D07) having NCBI having database Genbank gi 50745107 (SEQ ID NO:111), chromosome segregation protein smc1 (SEQ ID NO:119; Genbank gi 2135244), CPSF6 protein (SEQ ID NO:123; Genbank gi 12653847), DNA-activated protein kinase (Genbank gi 32140473) and DNA-activated protein kinase catalytic subunit (SEQ ID NO:126; Genbank gi 38258929), activating signal cointegrator 1 complex subunit 3-like 1 (SEQ ID NO:130; Genbank gi 40217847), heterogeneous nuclear ribonucleoprotein M (SEQ ID NO:133; Genbank gi 55977747), a protein similar to small nuclear ribonucleoprotein D1 having NCBI database Genbank gi 34877889 (SEQ ID NO:142), histone deacetylase 2 (SEQ ID NO:144; Genbank gi 4557641), histone H1b ((SEQ ID NO:146; Genbank gi 356168), histone H2A.5 (SEQ ID NO:152; Genbank gi 70686), a protein similar to histone H3 having NCBI database Genbank gi 30156584 (SEQ ID NO:153), histone H3 (SEQ ID NO:158; Genbank gi 386772), histone H4 (SEQ ID NO:159; Genbank gi 223582), nucleolar protein NOP5/NOP58 (SEQ ID NO:162; Genbank gi 17380155; Genbank gi 21595782), PTB-associated splicing factor (SEQ ID NO:168; Genbank gi 38458), or SmB/B′ autoimmune antigen ((SEQ ID NO:180; Genbank gi 36495).

In addition or in the alternative, detection of biomarkers can be detection of the proteins of Table 2 identified as downregulated proteins that are underexpressed by 5 fold or greater in cancer cells, such as one or more of annexin V chain c (SEQ ID NO:7; Genbank gi 809190), epididymal protein (SEQ ID NO:17; Genbank gi 23092553), glutaminase (SEQ ID NO:19; Genbank gi 12044394), inter-alpha-trypsin inhibitor heavy chain 3 precursor (SEQ ID NO:30; Genbank gi 3024064), alpha 2 macroglobulin (SEQ ID NO:29; Genbank gi 224053), membrane alanine aminopeptidase precursor (SEQ ID NO:31; Genbank gi 37590640; Genbank gi 4502095), type-2 phosphatidic acid phosphohydrolase (SEQ ID NO:33; Genbank gi 3015569), pregnancy zone protein precursor (SEQ ID NO:36; Genbank gi 131756), transglutaminase 2 isoform a/transglutaminase C (SEQ ID NO:42; Genbank gi 39777597), or the unnamed protein product having a sequence with NCBI database Genbank gi 7023123 (SEQ ID NO:47).

### III. Identification of Phosphoproteins

The present invention also includes the use of SILAC/MS to investigate proteins that undergo posttranslational modifications in cells, such as, for example, glycosylated proteins, acylated or isoprenylated proteins, oxidized proteins, and phosphorylated proteins. In many cases proteins having particular modifications can be isolated from a cell lysate using affinity reagents, such as but not limited to antibodies, that specifically recognize the modified forms of the proteins. Cells can optionally be fractionated prior to isolation of proteins. Alternatively or in addition, proteins can be separated from cell lysates or cell fractions using protein separation methods such chromatography, electrophoresis, differential precipitation, differential extraction, etc.

Of particular interest are phosphoproteins. Phosphorylation and dephosphorylation regulate many cellular events, such as cell cycle control, cell differentiation, transformation, apoptosis, signal transduction, etc. The present invention provides methods for studying phosphorylation cascades by identifying proteins whose phosphorylation state can be altered by a chemical, biological or even physical perturbation, such as, for example, drug treatment, toxic insult, physical exposure (such as radiation or UV treatment) or stimulation with growth or differentiation factors, etc.

One aspect of the present invention is a method for isolation one or more phosphoproteins or phosphopeptides that comprises applying a cell fraction comprising a phosphoprotein or phosphopeptide to a column, in which the column contains a matrix having a resin derivatized with iminodiacetic acid (IDA) that has been charged with Fe⁺³; and eluting one or more phosphoproteins or phosphopeptides from the column to obtain one or more isolated phosphoproteins or phosphopeptides. Preferably, a preparation of peptides (for example, protease digested proteins) is applied to the IDA column charged Fe⁺³ with for the isolation of phosphopeptides.

For example, the column can comprise a resin derivatized with iminodiacetic acid (IDA) that has a ligand density of from about 10 to about 60 micromoles per milliliter, preferably a ligand density of from about 20 to about 50 micromoles per milliliter, more preferably having a ligand density of from about 20 to about 50 micromoles per milliliter, and even more preferably of from about 30 to about 40 micromoles per milliliter. In some preferred embodiments, the resin has a ligand density of about 35 micromoles per milliliter. The resin particles preferably have a particle size of from about 30 microns to about 100 microns in diameter, more preferably from about 40 microns to about 90 microns in diameter, more preferably yet from about 50 microns to about 80 microns in diameter, and even more preferably of about 65 microns in diameter.

A preferred column for the isolation of phosphoproteins or phosphopeptides is a column that contains the resin made by Tosoh Biosciences, and designated Toyopearl AF-Chelate 650M resin (Tosoh Bioscience, Montgomery, Pa., 2005 part numbers 14475, 19800, and 14907) in which the resin is charged with a metal, such as but not limited to, Ga, Nb, Ti, Va, Ta, Sc, Al, Y. Zr, Ru, In, or Fe. Preferably, the column is charged with F3⁺. Fe³⁺.

In other embodiments, phosphoproteins or phosphopeptides can be isolated using Dyanbeads® Talon™ beads charged with Fe³⁺ (Invitrogen, Carlsbad, Calif.), a nitroloacetic acid (NTA) column charged with Fe³⁺, a Varian (Palo Alto, Calif.) Nexus column charged with Fe³⁺, or a column using Titansphere (TiO₂) available from GL Sciences Inc. (Japan).

A cell fraction can be a cell sample that has been lysed and preferably subjected to at least one separation procedure, including but not limited to: extraction with one or more solutions comprising at least one of a salt, a detergent or surfactant, an acid, or a base; centrifugation; solubilization; precipitation; affinity capture; dielectrophoresis; or chromatography. In some embodiments, phosphoproteins are first enriched by affinity capture using a specific binding reagent that binds one or more of phosphoserine, phosphothreonine, or phosphotyrosine. Proteins also can be separated from the cell fraction by methods such as electrophoresis or chromatography. In some preferred embodiments, proteins are digested with one or more proteases prior and the resulting peptide mixture is applied to the column.

In using a column that comprises IDA resin, such as Toyopearl AF-Chelate 650M, the matrix is preferably equilibrated in an acidic solution prior to applying a cell fraction to the column. The acidic solution preferably has a pH of from about 2 to about 4. A preferred acid is acetic acid at a concentration of from about 0.01% to about 1%, preferably at a concentration of from about 0.05% to about 0.5%, and more preferably at a concentration of about 0.1%.

After binding of phosphoproteins or phosphopeptides, the matrix can be washed with one or more wash solutions comprising at least one acid. Elution of phosphoproteins or phosphopeptides can be in a solution that comprises one or more of piperidine, imidazole, a solution that contains o-phosphate, or a solution that has a basic pH, preferably a pH of between about 8 and about 11, or between about 8.5 and about 10.5., or a pH of about 9. Some preferred elution buffers comprise, for example, ammonium carbonate, ammonium hydroxide, diammonium citrate, ammonium acetate, ammonium dihydrogen phosphate, or ammonium bicarbonate. Another preferred solution for elution of phosphopeptides is a mixture of 2,5-dihydroxybenzoic acid and o-phosphoric acid.

One preferred protocol for the enrichment of phosphopeptides with immobilized metal affinity chromatography (IMAC) is the following: Toyopearl AF-Chelate-650M resin (2 to 4 μl suspension) is packed into an Eppendorf GELoading tip (1-10 μl; Eppendorf AG, Hanmburg, Germany) with a home-made frit (e.g. a tiny sliver of Porex polyethylene sheet; Porex products, X 4900), and subsequently loaded with 100 mM FeCl₃ in 2% acetic acid. The tip column is washed with 40% acetonile, and equilibrated with 0.1% acetic acid. The tryptic peptides extracted from gel pieces are dried in Speed Vac, resuspended in 50 μl of 10% acetonitrile in 0.1% acetic acid. The sample is pushed slowly through the tip column using an Eppendorf Repeater pipettor without allowing the column to dry. The tip column is washed sequentially with 30 μl of 0.1% acetic acid, 30 μl of 25% acetonitrile in 0.1% acetic acid, and then eluted with 6 to 10 μl of 100 mM ammonium bicarbonate (pH 9.0). The eluted phosphopeptides may be dried in a Speed Vac (or carefully lyopholyzed) and resuspended in 2 μl of 10% acetonitrile in 0.1% TFA or 0.1% formic acid for direct MS analysis, or abundant protein digests may be desalted with a C₁₈ ZipTip column and subsequently eluted with 75% acetonitrile in 0.1% TFA.

Phosphoproteins or phosphopeptides can be analyzed by mass spectrometry to obtain a mass spectrometry profile, for example, using LC-MS/MS, nanoelectospray MS/MS, or MALDI-TOF to identify one or more phosphoproteins or phosphopeptides.

SILAC/MS with Kinase Inhibitor and Phosphoprotein/Peptide Isolation

The invention also includes a method of identifying a protein whose phosphorylation in cells is inhibited by a kinase inhibitor, where the method includes: providing a first cell culture comprising cells and at least one isotope at a non-natural level in a from that is metabolically incorporated into proteins within cultured cells; providing a second cell culture comprising cells that do not comprise the at least one isotope at a non-natural level in the cell media; adding a kinase inhibitor to the first culture; allowing the cells in each of the cell cultures to divide;

combining at least a portion of the cells of the first with at least a portion of the cells of the second cell culture to form a mixed cell sample; separating one or more phosphoproteins from the mixed cell sample; performing mass spectrometry on the one or more proteins; identifying phosphoproteins in said one of the one or more proteins comprising the non-naturally occurring isotope with the abundance of the corresponding naturally-occurring isotope; and identifying at least one protein whose phosphorylation in cells is inhibited by a kinase inhibitor as a phosphoprotein that is present in greater abundance in the second cell culture than in the first cell culture.

The method can use media having one or more heavy isotope labels for one of the cultures being compared. For example, heavy Lysine (U-13C₆-Lysine) and heavy Arginine (U-13C₆-Arginine) are useful where proteins are digested with trypsin for MS analysis. Heavy tyrosine (U-¹³C₉-Tyrosine) is also an option for isotopic label, especially for analysis of phosphorylated peptides. Double labeled isotopes can also be used, for example heavy Arginine (U-15N, U-13C₆-Arginine) can be combined with heavy Lysine (U-13C₆-Lysine) in a media formulation.

The method can be used to identify one or more phosphoproteins whose phosphorylation is inhibited by a particular drug or compound. Such identified phosphoproteins can be candidate drug targets.

In yet another aspect, the invention provides a peptide derived from the Dok-2 protein that has a tyrosine residue that is phosphorylated in CML cells, where the peptide comprises the sequence GQEGEYAVPFDAVAR (SEQ ID NO:186). The tryptic peptide spans amino acids 294 through 318 of the human Dok-2 protein (SEQ ID NO:187; NCBI Genbank gi 41406050), and includes the tyrosine residue that is phosphorylated in CML cells is residue 299 of the protein sequence as listed in Genbank gi 41406050 in the NCBI database. The peptide so identified will hereinafter be referred to as “Dok-2/294-318”.

The invention also includes peptides and proteins having sequences homologous to at least a portion of the human Dok-2/294-318 sequence, and include the Tyr 299 residue. Such homologous sequences can be from any source, and can be designed and chemically synthesized or genetically engineered and expressed either as peptides or proteins. The sequences can be, for example, obtained by random, semi-random, or nonrandom mutation, or derived from proteins such as but in no way limited to sequences of Dok-2 proteins or Dok-2 homologs of other species. The peptides comprising sequences homologous to at least a portion of the human Dok-2/294-318 sequence can be phosphorylated to a detectable level by Bcr-Abl kinase or by other kinases that are directly or indirectly regulated by the Bcr-Abl kinase in cells, or by other kinases that are directly or indirectly inhibited by ST1571 in cellular assays.

The homologous sequences preferably are at least 50%, at least 60%, at least 70%, at least 75%, at least 80%, at least 85%, at least 90%, at least 95% or at least 99%. As used herein, “homologous” also includes 100% homologous, i.e., identical. The percent homology between two sequences can be determined using sequence analysis software. Such software matches similar sequences by assigning degrees of homology to various insertions, deletions, substitutions, and other modifications. Exemplary software include: The Sequence Analysis Software Package of the Genetics Computer Group, University of Wisconsin Biotechnology Center, Madison, Wis.; (Devereux et al., Nucleic Acids Res. 12:387, 1984) The algorithm of E. Myers and W. Miller (CABIOS, 4:11, 1989), which has been incorporated into the ALIGN program (version 2.0), using a PAM120 weight residue table, a gap length penalty of 12 and a gap penalty of 4; The NBLAST and XBLAST programs (version 2.0) of Altschul et al. (J. Mol. Biol. 215:403, 1990).

BLAST nucleotide searches can be performed with the NBLAST program, score=100, wordlength=12 to obtain nucleotide sequences homologous to the nucleic acid molecules of the invention. BLAST protein searches can be performed with the XBLAST program, score=50, wordlength=3 to obtain amino acid sequences homologous to the proteins of the invention. To obtain gapped alignments for comparison purposes, Gapped BLAST can be utilized as described in Altschul et al. (Nucleic Acids Res. 25:3389, 1997). When utilizing BLAST and gapped BLAST programs, the default parameters of the respective programs (e.g., XBLAST and NBLAST) can be used.

The invention also includes peptides and proteins comprising the human Dok-2 294-318 sequence, or sequences homologous to the human Dok-2 294-318 sequence, that can be phosphorylated by the Bcr-Abl kinase, or that can be phosphorylated by one or more other kinases that are inhibited by ST1571, or that can be phosphorylated by one or more other kinases that are directly or indirectly regulated by the Bcr-Abl kinase or by one or more kinases inhibited by ST1571.

The invention includes uses of the Dok-2 294-318 peptide, peptides homologous to the Dok-2 294-318 peptide, peptides and proteins comprising the Dok-2 294-318 peptide, and peptides and proteins comprising sequences homologous to the Dok-2 294-318 peptide (hereinafter referred to as Dok-2 294-318 peptide homologous peptides and proteins) in kinase and phosphatase assays.

Kinase assays can be conducted in any format, for example, the Dok-2 294-318 peptide homologous peptide or protein can be bound to a solid support, and detection of labeled phosphate bound to the solid support can indicate kinase activity, or the assay can be performed with the substrate Dok-2 294-318 peptide homologous peptide or protein in solution, where the Dok-2 294-318 peptide homologous peptide or protein is subsequently captured, precipitated, or separated, for example, on a column or gel, for detection of incorporated phosphorous.

Kinase assays (including assays for inhibitors of kinases) in which the Dok-2 294-318 peptide homologous peptides and proteins are used as substrates, and phosphorylation of the tyrosine residue corresponding to Tyr 299 of Dok-2 is detected can use detectable labels to monitor incorporation of phosphate into the peptide. Detection methods for kinase activity are known in the art, and include, but are not limited to, the use of labels, such as but not limited to radioactive labels (e.g., ³³P-ATP and ³⁵S-g-ATP), or probes, such as antibody probes that bind to phosphoamino acids. Antibody probes can be labeled, or detection can be by means of a secondary binding member that comprises a detectable label. The methods include providing a peptide or protein comprising a sequence homologous to at least a portion of the human Dok-2/294-318 sequence (GQEGEYAVPFDAVAR (SEQ ID NO:186)); contacting the peptide or protein with at least one cell extract or at least one kinase and at least one phosphorous-containing compound that can be used as a substrate by at least one kinase; detecting the incorporation of phosphorous into the peptide or protein.

In some embodiments, the peptide or protein can comprise the human Dok-2/294-318 sequence (GQEGEYAVPFDAVAR (SEQ ID NO:186)) and additional sequences unrelated to human Dok-2 sequences. In other embodiments, the assays can use the human Dok-2/294-318 peptide. In yet other embodiments, the assays can use a peptide or protein that comprises sequences homologous but not identical to the human Dok-2/294-318 sequence.

A cell fraction or a partially or substantially purified kinase can be used in the assays. The kinase can be any kinase, such as Bcr-Abl or another kinase inhibited by ST1571, or an unrelated tyrosine kinase. In most cases, ATP is provided as a source of phosphate, but other phosphorous-containing compounds that can be used by a kinase can be used. The phosphorous compound can be labeled for the detection of phosphorylation, or phosphorylation can be detected by the use of directly or indirectly labeled antibodies. For example, a secondary antibody can be used for sandwich hybridization.

Similarly, phosphatase assays, as well as assays for inhibitors of a phosphatase, can be conducted using Dok-2 294-318 peptide homologous peptides and proteins. For example, whereas incorporation into a protein of labeled phosphorus indicates kinase activity in one assay, another assay can be used to measure the reduction of labeled phosphorous on a solid support or captured substrate, or by the release of labeled phosphorus into the media, indicating phosphatase activity. Phosphatase assays can be performed by providing a peptide or protein comprising a sequence homologous to at least a portion of the human Dok-2/294-318 sequence (GQEGEYAVPFDAVAR); contacting the peptide or protein with at least one cell extract or at least one phosphatase; and detecting the release of phosphorous from the peptide or protein.

The following examples are intended to illustrate but not limit the invention.

### Example 1

### Identification of Biomarkers for Breast Cancer

More than 50% of all major drug targets are membrane proteins and their role in cell-cell interaction and signal transduction is a vital concern. Based on labeling of normal and malignant breast cells with light or heavy isotopes of amino acids (SILAC), cell fractionation and ID gel separation of crude membrane proteins, followed by analysis of nanoelectrospray LC-MS/MS, we have quantified over 1600 proteins with approximate 1000 membrane-associated proteins and 250 unknown or hypothetical proteins. A number of proteins show increased expression levels in malignant breast cells, such as autoantigen p542, osteoblast-specific factor 2 (OSF-2), 4F2 heavy chain antigen, 34 KD nucleolar scleroderma antigen, and apoptosis inhibitor 5. The expression of other proteins, such as membrane alanine aminopeptidase (CD13), epididymal protein, macroglobulin alpha2, and transglutaminase C, decreased in malignant breast cells, whereas the majority of proteins remained unchanged when compared to the corresponding non-malignant samples. Downregulation of CD13 and upregulation of OSF-2 was validated and confirmed with human tissues with breast carcinomas using immunohistochemistry. These results indicated that SILAC is a powerful technique for that can be extended to biomarker discovery.

Stable isotope labeling with amino acids in cell culture (SILAC) is a simple and accurate approach to quantify differential protein expression and dynamic regulation of posttranslational modificationl-10. Two populations of cells are grown in identical media except that one contains light amino acids and the other contains heavy amino acids. For example, ¹³C-labeled amino acids, such as [U-¹³C₆]Lys and [U-¹³C₆]Arg, are stable isotopes and can be handled like regular amino acids. During cell culturing, light or heavy amino acids are incorporated into proteins using the natural biosynthetic machinery of the cells. The labeling efficiency is almost 100%. Cells labeled with light or heavy amino acids are combined and treated as a single sample prior to any process or protein purification, eliminating quantification error due to unequal sample preparation and increasing reproducibility. When dual labels of heavy Lys and Arg are used, all tryptic peptides carry a mass tag with exception of C-terminal peptides, because trypsin cleaves polypeptides after Lys or Arg. Proteins and peptides labeled with light or heavy amino acids are chemically identical and co-elute in any liquid chromatography or electrophoretic separations. Nevertheless, the mass difference between the light and heavy peptides is distinguishable by mass spectrometry. Once isotopic peptide pairs have been correlated with the proteins from which they originate, their relative intensities can be used to quantify different expression of the parent protein between normal and disease state cells.

Membrane proteins play a pivotal role in regulating cell-cell interaction, recognition, migration, adhesion, and signal transduction. Currently more than 50% of all major drug targets for medicines are membrane proteins. In this report, we describe a SILAC approach in the quantification of differential membrane expression between normal and malignant breast cells from cell lines derived from a 74-year-old female with breast carcinoma.

### Methods

NuPAGE gel, NuPAGE sample buffer, SimplyBlue SafeStain, Invitromass LMW calibrants, DMEM medium, Lys and Arg-deprived DMEM medium, FBS, dialyzed FBS, epidermal growth factor (EGF), SuperPicTure™ Polymer Detection Kit, and monoclonal anti-CD13 antibody were obtained from Invitrogen Life Sciences. [U-¹³C₆] L-Lysine and [U-¹³C₆] L-Arginine were purchased from Cambridge Isotope Laboratories. Normal L-Lysine, L-Arginine, Aprotinin, Leupeptin Hemisulfate, phenylmethanesulfonyl fluoride (PMSF), and insulin were purchased from Sigma. Rabbit anti-osteoblast specific factor 2 (OSF-2) antibody was ordered from Biovendor Laboratory Medicine, Inc. Trypsin was ordered from Promega. Benzonase was from Novagen. Normal (HTB-125) and malignant (HTB-126) breast cells, isolated from a 74 female with breast carcinoma, were purchased from ATCC.

Normal breast cells were maintained in DMEM medium containing 10% FBS and 30 ng/ml EGF and malignant cells were maintained in DMEM medium containing 10% FBS and 30 ng/ml insulin. For labeling of cells with light or heavy amino acids, aliquots of normal and malignant breast cells (105 cells each) were harvested separately. Normal breast cells were resuspended in 3 ml of modified DMEM medium supplemented with 10% dialyzed FBS, 30 ng/ml EGF, light L-Lysine and light L-Arginine, whereas malignant cells were resuspended in 3 ml of modified DMEM medium supplemented with 10% dialyzed FBS, 30 ng/ml insulin, heavy [U-¹³C₆] L-Lysine and heavy [U-13C₆] L-Arginine. Initially normal and malignant cells were cultured in two separate 60 mm dishes. Every three to four days, either the media was replaced or the cells were split with the media being replenished with the corresponding light medium for normal cells or heavy labeling medium for malignant cells. Normal (˜10⁶ cells/100 mm dish) and malignant (˜10⁶ cells/100 mm dish) breast cells labeled with light or heavy amino acids are scraped off the dishes and mixed at 1:1 ratio. The cell mixture was lysed on ice for 30 min in 1.6 ml of hypotonic buffer (10 mM Tris-HCl (pH 7.4), 1 mM MgCl₂, 0.5 mM PMSF, 0.15 μM Aprotinin, 1 μM Leupeptin Hemisulfate, and 10 U/ml benzonase), followed by 30 strokes of a Dounce homogenizer. To the cell suspension, 0.4 ml of 5× sucrose (1.25 M sucrose stock in H₂O) was added and mixed five times, followed by centrifugation at 500×g for 10 min to remove nuclei. The supernatant was then centrifuged at 100,000×g for 1 h to obtain a crude membrane fraction. Membrane pellets were dissolved in 60 μl of 2×SDS sample buffer containing 50 mM DTT, heated at 95° C. for 5 min, and half of the sample was analyze by SDS-PAGE. The entire gel lane was cut into 30-45 fractions and the gel pieces were subjected to in-gel tryptic digest.

Tryptic peptides labeled with light or heavy amino acids were analyzed with nanoelectrospray LC-MS/MS on a Q-TOF API-US instrument (Waters Corporation). Atlantis™ dC18, 3 μm, 100 μm×100 mm column (Waters Corporation) was used for peptide separation. Alternatively, peptides were fractionated and spotted onto MALDI plates in a Nano LC-Probot system (LC Packings, Dionex), followed by the analysis with a 4700 proteomics analyzer (Applied Biosystems). For simple digests, a gradient of 5-45% (v/v) acetonitrile in 0.1% formic acid over 45 min, and then 45-95% acetonitrile in 0.1% formic acid over 5 min was used. For a complex sample, a gradient of 5-45% (v/v) acetonitrile over 90 min, and then 45-95% acetonitrile over 30 min was used. For a very complex sample, a gradient of 5-45% (v/v) acetonitrile over 120 min, and then 45-95% acetonitrile over 60 min was used. On the Q-TOF, four components were used to acquire MS/MS data with 1.4 s scan time. On the 4700 proteomics, peptides with mass over 1000 were chosen for MS/MS analysis.

Raw data files from Q-TOF instrument were processed with Mascot Distiller (Matrix Science, London) and then searched against NCBI database using Mascot search algorithm. A pair of light and heavy Lys and a pair of light and heavy Arg with delta mass of 6 Da were selected for variable modification. The mass tolerance of the precursor peptide ion was set at 200 ppm and mass tolerance for the MS/MS fragment ions was set at 0.5 Da. The Mascot output shows peptides labeled with light or heavy Lys and/or Arg. Quantification of peptide pairs was done and validated manually by examining both MS and MS/MS spectrums.

Raw data files from 4700 Proteomics were processed using GPS explorer (Applied Biosystems). Quantification software for ICAT works equally well on SILAC, except that the delta mass for SILAC is 6 Da for a pair of light and heavy Lys or Arg. At this moment, only one pair of light and heavy Lys or Arg can be selected for quantification. The Mascot search result can show identities of proteins as well as the relative ratio of isotopic peptide pairs.

Immunohistochemistry. Human tissue samples were embedded in paraffin. Slides with tissue sections were deparaffinized in xylene and dehydrated with alcohol. Antigens were retrieved with either 0.01 M citrate (pH 6.0) or 0.1% Trypsin-EDTA (pH 8.0) buffer. After incubating primary anti-OSF-2 antibody at a concentration of 0.125 μg/ml or anti-CD13 antibody at 1:50 dilution for 1 h, the HRP polymer was added and DBA chromogen was used for detection.

### Results

Shown is FIG. 1 is the strategy used to identify and quantify differential expression of membrane proteins between normal epithelial and malignant breast cells isolated from a 74 year old female with breast carcinoma. Normal and malignant breast cells were grown in DMEM medium supplemented with either light Lysine and Arginine or heavy [U-¹³C₆]Lysine and [U-¹³C₆]Arginine, respectively. In a typical experiment, both Lys and Arg were uniformly labeled with heavy atoms of carbon C-13 making the amino acids 6 Da heavier than the light or naturally abundant carbon C-12 Lys and Arg. After propagating the cultures for at least six doubling times, aliquots of cells labeled with either light or heavy amino acids were lysed separately and analyzed by SDS-PAGE side by side. Protein bands with light or heavy labels were excised from the side by side gel lanes and subjected to in-gel tryptic digest. The tryptic peptide extracts with either light or heavy label were analyzed in parallel using MALDI-TOF. A complete shift of 6 Da for peptides containing heavy Lys and/or Arg was observed, indicating that 100% incorporation of heavy Lys and/or Arg into proteins was achieved.

In experiments to identify proteins differentially expressed between normal and malignant breast cells, normal and malignant cells were labeled with light or heavy amino acids as described above, scraped off the dishes, counted, and combined at a 1:1 ratio. The cell mixture (typically 3×10⁶ total cells) was lysed in 1.6 ml of hypotonic buffer (10 mM Tris-HCl, 1 mM MgC12, Benzonase (10 U/ml), 0.5 mM PMSF, 0.15 uM Aprotinin, and 1 uM Leupeptin Hemisulfate) on ice for 30 min, followed by 30 strokes of a dounce homogenizer. 0.4 ml of 1.25 M sucrose was added to the cell suspension, which was dounced 5 more times. After removing nuclei by centrifugation at 500×g for 5 min, crude membrane pellets were obtained by ultracentrifugation for 100,000×g for 1 hour. Crude membrane pellets were directly dissolved in SDS sample buffer, followed by the analysis of SDS-PAGE. The entire gel lane was divided into approximately 40 sections, and then the proteins were in-gel digested with trypsin. The resulting peptide extract was analyzed by nESI LC-MS/MS on the Q-TOF instrument.Up to 100 proteins could be identified in a single LC-MS/MS run. Raw data was processed with Mascot Distiller. Protein identifications were performed with the Mascot search algorithm and quantification was achieved by manual analysis of corresponding peptide pairs in the MS spectrum.

As expected, isotopic peptide pairs always coeluted. For nanoelectrospray LC-MS/MS analysis on the Q-TOF instrument, the charge states of the peptides mostly appear at +2 and +3, although occasionally at +4. Therefore, in the MS spectrum, most of the peptide pairs are 3, 2, or 1.5 Da apart, respectively. Sometimes both light and heavy peptides were triggered for MS/MS fragmentation. In that case, some of their daughter ions, especially b fragment ions, were identical, but some y fragment ions were 6 Da apart as the daughter ions are singly-charged. Up to 25 isotopic peptide pairs derived from the same protein were recovered in a single LC-MS/MS run. Quantification was achieved through the average of multiple peptide pairs derived from the same protein. For example, we recovered nine peptides from vimentin, including peptides containing two lysines and/or arginines. The relative ratio of those isotopic peptide pairs was consistent and the standard deviation was ±10% (FIG. 2). The ratio of isotopic peptide pairs was consistent regardless of the number of lysine and/or arginine in the peptides or the charge state of the peptides. For example, the relative ratio of isotopic peptides of LQDEIQNMKEEMAR (SEQ ID NO:193) at charge state +2 was almost identical to that at charge state +3.

In cases where peptides or peptide pairs overlapped, the overlapping peptides were not used for quantification. Instead, quantitation for each individual protein that exhibited an overlapping peptide was achieved through examining other peptide pairs derived from the protein.

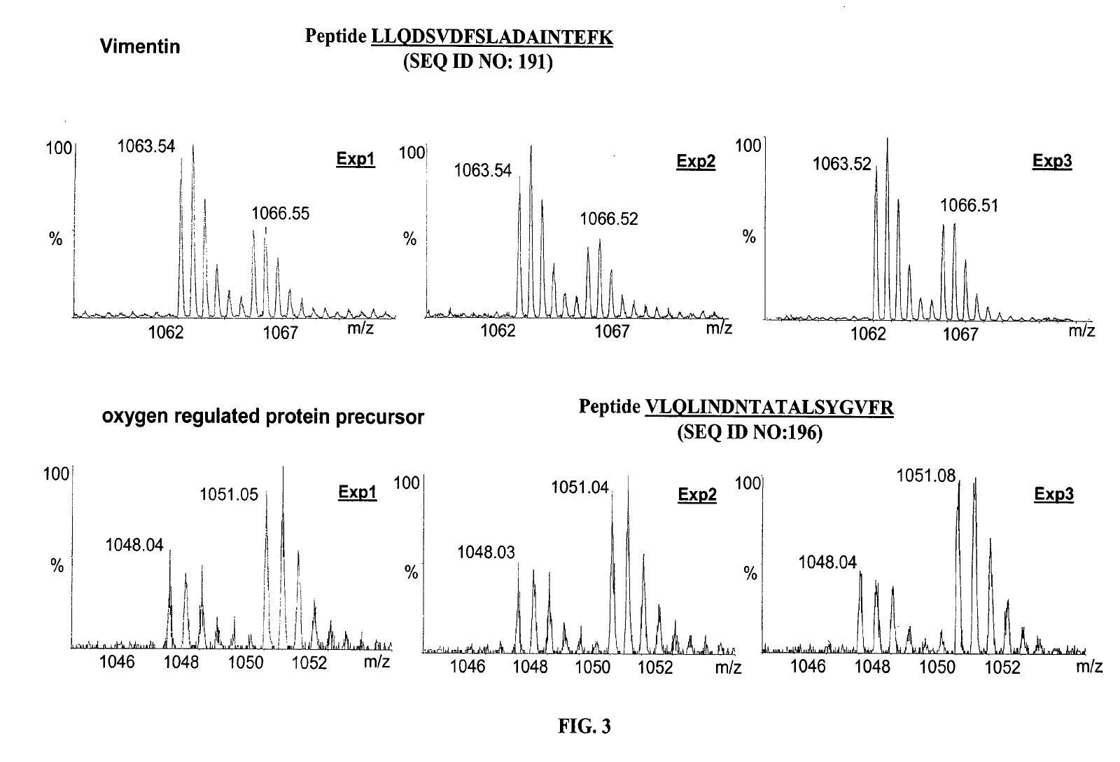

Unlike other chemical labeling methods, which handle two samples separately until the last step of analysis, SILAC mixes two populations of cells in the first step and then the cell mixture is treated as a single sample in all the subsequent steps. Therefore, SILAC is more accurate and reproducible. We performed three individual experiments, and found that as long as the cell number from normal and malignant cells was normalized, the results were highly reproducible. The same peptide of LLQDSVDFSLADAINTEFK (SEQ ID NO:191) derived from vimentin and peptide of VLQLINDNTATALSYGVFR (SEQ ID NO:196) derived from oxygen regulated protein precursor were recovered from three separate experiments. The relative ratio of heavy over light peptide was around 0.5 for vimentin, and was around 2.0 for oxygen regulated protein at all three separate experiments. The relative ratio of those isotopic peptide pairs for each individual protein remained remarkably constant (FIG. 3), with a standard deviation of +10%.

Quantitation of peptides containing proline required special consideration. This is because amino acids provided in the culture medium are subject to cellular metabolism. arginine can be converted to proline via a glutamic acid γ-semialdehyde intermediate. The percentage of conversion is likely dependent on the cell type used as well as the amount of arginine and proline in the culture medium. The conversion of ¹³C arginine to ¹³C proline results in the presence of an additional satellite peak that shadows proline-containing peptides arising from cells grown in heavy ¹³C arginine media. Because proline retains five ¹³C atoms, these heavy isotopic forms of proline-containing peptides appear at 2.5 and 1.6 m/z units, respectively, above the double and triple charge states of the most abundant form of the Lys and/or Arg-labeled peptide. That is to say, although the incorporation of Arg is essentially complete, the conversion of arginine to proline splits that mass signal stemming from proline-containing peptides of the heavy-labeled cell state into two channels. Accordingly, when proline-containing peptides are used for quantification, a correction factor in peak intensity for total heavy labeled peptides would be the sum of peak intensities of these mass channels.

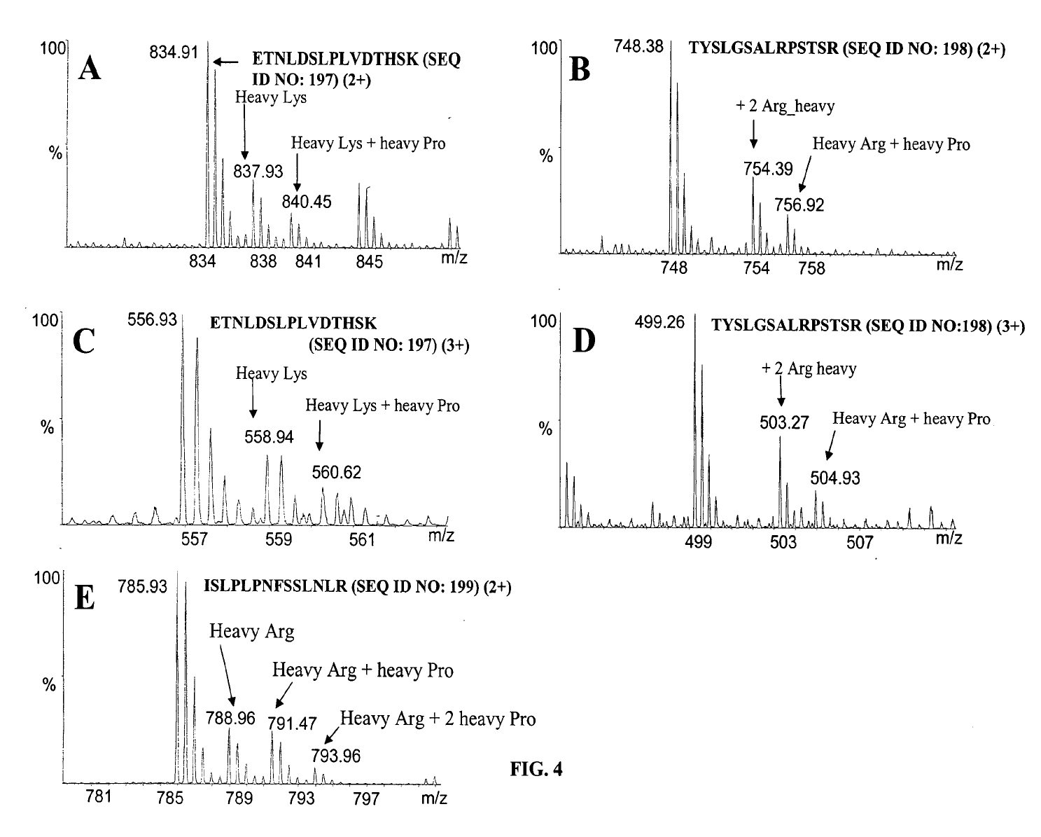

In FIG. 4, satellite peaks are observed for proline-containing peptides because Arg can be converted into proline (Pro) via α-ketoglutarate intermediate. When labeling cells with [U¹³C₆]Arg, a small satellite peak appears at [M+2.5] Da if the peptide is doubly charged and at [M+1.6] Da if triply-charged since proline has five carbons. However, prolines arising from [U¹³C₆, ¹⁵N₄]Arg by metabolic interconversion are 6 Da heavier due to the incorporation of a heavy nitrogen isotope. The correct amount of peptides with heavy labels would be the sum of peptides with heavy Lys and/or Arg and peptides with heavy Lys and/or Arg plus heavy Pro.

For example, as depicted in FIG. 4A, the peak intensity for heavy labeled peptide ETNLDSLPLVDTHSK (SEQ ID NO:197) would be the sum of peptides at m/z 837.93 and at m/z 840.45, although the peak intensity for light peptide remains at m/z 834.91. The correction ‘rule’ is independent of the charge state of the peptide as well as the number of lysines and/or arginines in the peptides. Because membrane proteins, especially matrix proteins, often contain proline, the proline effect should be taken into consideration when the relative ratio of an isotopic peptide pair is calculated.

Quantification becomes very complicated if peptides contain multiple prolines. For example, as depicted in FIG. 4E, a peptide of ISLPLPNFSSLNLR (SEQ ID NO:199) derived from vimentin was recovered. The intensity of heavy labeled peptides would be the sum of peptides at m/z 788.96, 791.47, and 793.96. Where four of more peptides that do not contain proline were recovered, the corresponding proline-containing the peptides were ignored. Because of the difficulty in quantitating proline-containing peptides, in general, we would not recommend that proline-containing peptides be used for calculating quantitative ratios when cells are labeled with heavy arginine.

Using SILAC approach, we have identified and quantified more than 1600 proteins, including approximate 1000 membrane or membrane-associated proteins, 250 unknown or hypothetical proteins, and 350 ribosomal, heated-shock, or histone proteins. The majority of proteins remained unchanged when compared to the corresponding non-malignant samples. Proteins with differential expression larger than 2-fold are displayed in Table 1. Proteins with differential expression larger than 3-fold are displayed in Table 2. Certain cell-adhesion or matrix proteins, such as epican, vimentin, integrin beta 1 isoform 1A precursor, annexin 1, A2, VI, and especially annexin V show decreased expression levels in malignant breast cells. Proteins regulating the function of ion channels and transporters, such as stomatin, plasma membrane calcium-transporting ATPase, and vesicle amine transport protein 1, showed decreased expression in cancer cells. Alpha 2 macroglobulin (A2M), a large glycosylated protease inhibitor, showed significant decrease in malignant breast cells. has been shown to be differentially expressed in normal prostatic stromal and epithelial cells, with increased expression in the stroma surrounding prostate cancer cells and undetectable expression in most tumor cells (Bogenrieder T et al. Prostate 33:225-32, 1997). A 10 fold downregulation of membrane alanine aminopeptidase precursor (CD13), a zinc-dependent metallopeptidase, was observed in breast cancer cells in this SILAC study. We further examined the expression of CD13 in normal and neoplastic human breast tissues using immunohistochemistry. The immunoreactivity patterns and statistics obtained with tissue sections from breast carcinomas suggest that expression of CD13 is significantly decreased in breast carcinomas, which is in agreement with the result obtained from mass spectrometry.

TABLE 1
Proteins Having a Difference in Abundance of 2-Fold or
Greater between Breast Cancer Cells and Normal Cells

| | NCBI GI | Quanti- | SEQ ID |
|---|---|---|---|
| | number | tation | NO |
| Downregulated proteins: | | | |
| Membrane or membrane-bound proteins | | | |
| ATPase | 291868 | 2 | 1 |
| Annexin I | 442631 | 2.4 | 2 |
| Annexin A2 | 50845387 | 2.7 | 3 |
| Annexin A2, isoform 2 | 18645167 | 2.2 | 4 |
| Human Annexin V With Proline Substitution By Thioproline | 3212603 | 2.8 | 5 |
| annexin VI isoform 1; | 4502109 | 2.2 | 6 |
| Chain C, Annexin V | 809190 | 5.5 | 7 |
| Similar to cytoskeleton-associated protein 4 [H. sapiens] | 19263767 | 2.1 | 8 |
| CD81 antigen | 12804239 | 3.7 | 9 |
| CD107a antigen | 126376 | 2.5 | 10 |
| CD109 | 19071209 | 2 | 11 |
| Delta-sarcoglycan (SG-delta) (35 kDa dystrophin-associated | 13431864 | 2.2 | 12 |
| glycoprotein) | | | |
| 1-8D | 23396 | 3.4 | 13 |
| Epican | 31191 | 2 | 14 |
| Erythrocyte band 7 integral membrane protein (Stomatin) | 114823 | 2.7 | 15 |
| endoglin precursor | 182091 | 2.1 | 16 |
| epididymal protein | 23092553 | 8.6 | 17 |
| growth regulated nuclear 68 protein | 226021 | 2 | 18 |
| glutaminase | 12044394 | 5.5 | 19 |
| guanine nucleotide-binding regulatory protein alpha- | 183182 | 2 | 20 |
| inhibitory subunit | | | |
| GTP-binding protein Rab3B | 106187 | 2.6 | 21 |
| hexokinase 1 isoform HKI | 4504391 | 3.5 | 22 |
| integrin beta 1 isoform 1A precursor | 19743813 | 2.3 | 23 |
| integrin alpha 2 precursor; Integrin, alpha-2 | 4504743 | 2.8 | 24 |
| inter-alpha-trypsin inhibitor heavy chain 3 precursor | 3024064 | 5.3 | 25 |
| Lysosome-associated membrane glycoprotein 1 precursor | 126376 | 2.5 | 26 |
| (LAMP-1) | | | |
| lysosome-associated membrane protein-3 variant | 21070332 | 2.9 | 27 |
| membrane-type matrix metalloproteinase | 793763 | 4.3 | 28 |
| macroglobulin alpha2 | 224053 | 7.5 | 29 |
| Membrane alanine aminopeptidase, precursor | 37590640 | 5 | 30 |
| membrane alanine aminopeptidase precursor | 4502095 | 10 | 31 |
| Major vault protein | 19913410 | 2 | 32 |
| type-2 phosphatidic acid phosphohydrolase | 3015569 | 5.1 | 33 |
| Plasma membrane calcium-transporting ATPase 4 (PMCA4) | 14286105 | 2.5 | 34 |
| plasminogen activator inhibitor type 1, member 2; | 24307907 | 3.8 | 35 |
| Pregnancy Zone Protein Precursor | 131756 | 6.4 | 36 |
| protein PP4-X | 189617 | 2.4 | 37 |
| Similar to cytoskeleton-associated protein 4 | 19263767 | 2.5 | 38 |
| Structure specific recognition protein 1 | 4507241 | 2 | 39 |
| syntenin | 2795863 | 2.2 | 40 |
| band 7.2b stomatin | 1103842 | 2.7 | 41 |
| transglutaminase 2 isoform a; transglutaminase C; | 39777597 | 6.5 | 42 |
| Vimentin | 418249 | 2.2 | 43 |
| Vesicle amine transport protein 1 | 18379349 | 2.1 | 44 |
| Unknown | | | |
| Unamed protein product | 28678 | 2.9 | 45 |
| unnamed protein product | 35218 | 2.1 | 46 |
| unnamed protein product | 7023123 | 7.5 | 47 |
| unknown | 2852648 | 2.7 | 48 |
| KIAA1253 protein | 6382026 | 2.9 | 49 |
| KIAA0908 protein | 4240305 | 2 | 50 |
| Others | | | |
| 5′-nucleotidase | 23897 | 4 | 51 |
| Upregulated proteins: | | | |
| Membrane or membrane-bound proteins | | | |
| alpha 1 type III collagen; | 4502951 | 4 | 52 |
| alpha 3 type VI collagen isoform 1 precursor | 4758028 | 2.2 | 53 |
| alpha 3 type VI collagen isoform 3 precursor; | 55743102 | 3.2 | 54 |
| autoantigen p542 | 3334899 | 7.5 | 55 |
| apoptosis inhibitor 5 | 30583025 | 4.9 | 56 |
| B23 nucleophosmin | 825671 | 4.5 | 57 |
| Chain H, Cathepsin D At Ph 7.5 | 5822091 | 2.3 | 58 |
| Chain A, Synthetic Ubiquitin With Fluoro-Leu At 50 And 67 | 31615803 | 3.7 | 59 |
| Collagen alpha 1(I) chain precursor | 115269 | 2.3 | 60 |
| Chain D, Tsg101(Uev) Domain In Complex With Ubiquitin | 48425523 | 3.6 | 61 |
| Carbamoylphosphate synthetase 2/aspartate | 41351087 | 3 | 62 |
| transcarbamylase/ | | | |
| cathepsin B | 3929733 | 3 | 63 |
| cathepsin Z precursor; CTSZ | 3294548 | 2.3 | 64 |
| coproporphyrinogen oxidase | 433888 | 3.4 | 65 |
| cysteine protease | 1890050 | 3.3 | 66 |
| Ephrin type-A receptor 2 precursor | 125333 | 2.3 | 67 |
| 4F2 heavy chain antigen | 177216 | 4 | 68 |
| fibroblast growth factor 2-interacting factor | 12656083 | 2.8 | 69 |
| farnesyl-diphosphate famesyltransferase | 435677 | 3.3 | 70 |
| FBRL_HUMAN; 34 KD NUCLEOLAR SCLERODERMA | 3399667 | 8 | 71 |
| ANTIGEN | | | |
| putative G-binding protein | 3153873 | 3.1 | 72 |
| integrin beta 4 binding protein isoform a; | 4504771 | 3.4 | 73 |
| lysosomal proteinase cathepsin B | 181178 | 5 | 74 |
| matrin 3 | 6563246 | 4.5 | 75 |
| methylene tetrahydrofolate dehydrogenase 2 precursor | 5729935 | 4.3 | 76 |
| Nucleolin | 4885511 | 2.8 | 77 |
| Nucleolar protein NOP5/NOP58 | 21595782 | 7.5 | 78 |
| Osteoblast-specific factor 2 | 46576887 | 12 | 79 |
| Oxygen regulated protein (150 Kd) | 5453832 | 2.2 | 80 |
| Opa-interacting protein OIP2 | 2815604 | 4.5 | 81 |
| prostacyclin synthase | 3135115 | 2.5 | 82 |
| RAD50 homolog isoform 1; | 19924129 | 3.3 | 83 |
| SP-H antigen | 743447 | 5 | 84 |
| SWI/SNF-related matrix-associated actin-dependent | 18606276 | 4.5 | 85 |
| regulator | | | |
| SLC3A2 protein | 12804283 | 3 | 86 |
| serine (or cysteine) proteinase inhibitor, clade H, member 1 | 32454741 | 2.4 | 87 |
| precursor | | | |
| scaffold attachment factor B | 1213639 | 3.2 | 88 |
| Solute carrier family 1 | 12652633 | 2 | 89 |
| Solute carrier family 7 (cationic amino acid transporter, y+ | 27503713 | 3.3 | 90 |
| system), member 5 | | | |
| thyroid-lupus autoantigen p70; | 4503841 | 5 | 91 |
| transferrin receptor | 4507457 | 2.2 | 92 |
| Transducin beta-like 3 | 23241743 | 2.6 | 93 |
| Voltage-dependent anion channel VDAC3 | 5733504 | 2 | 94 |
| Unknown | | | |
| unnamed protein product | 21750187 | 4 | 95 |
| unnamed protein product | 32097 | 5.8 | 96 |
| unnamed protein product | 7022744 | 6.1 | 97 |
| unnamed protein product | 21749696 | 7.5 | 98 |
| unnamed protein product | 10434070 | 4.4 | 99 |
| unnamed protein product | 34228 | 2 | 100 |
| unknown | 9789023 | 3.9 | 101 |
| hypothetical protein | 21740236 | 3 | 102 |
| hypothetical protein | 21739574 | 4.2 | 103 |
| hypothetical protein | 13276691 | 4 | 104 |
| hypothetical protein | 21739884 | 4.1 | 105 |
| nucleolar protein NOP5 (NOP58) | 7512749 | 7.5 | 106 |
| Hypothetical protein FLJ10292 | 17380155 | 6.3 | 107 |
| Hypothetical protein FLJ12525 | 15778927 | 6.4 | 108 |
| Human mRNA, complete cds | 348239 | 7.5 | 109 |
| FTSJ3 protein [Homo sapiens] | 62914003 | 5 | 110 |
| PREDICTED: similar to RIKEN cDNA 0610009D07 | 50745107 | 8 | 111 |
| Putatative 28 kDa protein | 14249858 | 3 | 112 |
| MGC2477 protein | 12655159 | 4.4 | 113 |
| Others | | | |
| ATP-dependant DNA helicase II | 17512093 | 3.2 | 114 |
| BRIX | 19070357 | 4.9 | 115 |
| cleavage and polyadenylation specific factor 6, 68 kD | 5901928 | 4.1 | 116 |
| subunit; | | | |
| cleavage and polyadenylation specific factor 5, 25 kD | 5901926 | 4.8 | 117 |
| subunit; | | | |
| chromatin-specific transcription elongation factor large | 6005757 | 5 | 118 |
| subunit | | | |
| chromosome segregation protein smc1 | 2135244 | 6.1 | 119 |
| cell cycle regulatory protein | 87057 | 3.7 | 120 |
| Chain B, Crystal Structure Of The Ku Heterodimer Bound To Dna | 15825717 | 3.8 | 121 |
| chaperonin | 41399285 | 2.2 | 122 |
| CPSF6 protein | 12653847 | 6.2 | 123 |
| dJ319D22.1 (CDC5-like protein) | 10183618 | 2.7 | 124 |
| DDX17 protein | 12653635 | 4.2 | 125 |
| DNA-activated protein kinase, catalytic subunit - human | 38258929 | 6.8 | 126 |
| DNA topoisomerase (ATP-hydrolyzing) | 105857 | 3.1 | 127 |
| DEAH (Asp-Glu-Ala-His) box polypeptide 9 isoform 1; | 4503297 | 4.6 | 128 |
| E2IG3 | 6457340 | 3.3 | 129 |
| U5 snRNP-specific protein, 200-KD | 40217847 | 6.2 | 130 |
| U5 snRNP-specific protein | 39963074 | 4.3 | 131 |
| hnRNP U protein | 32358 | 4.5 | 132 |
| hnRNA-binding protein M4 | 55977747 | 10 | 133 |
| heterogeneous nuclear ribonucleoprotein R | 5031755 | 4.9 | 134 |
| heterogeneous nuclear ribonucleoprotein L | 4557645 | 4.3 | 135 |
| Heterogeneous nuclear ribonucleoproteins A2/B1 (hnRNP | 133257 | 5.6 | 136 |
| A2/hnRNP B1) | | | |
| heterogeneous nuclear ribonucleoprotein C isoform b; | 4758544 | 7.3 | 137 |
| heterogeneous nuclear ribonucleoprotein G - human | 542850 | 4.6 | 138 |
| PREDICTED: similar to Small nuclear ribonucleoprotein Sm | 50756613 | 6.5 | 139 |
| D3 | | | |
| similar to Small nuclear ribonucleoprotein Sm D2 | 17471847 | 6 | 140 |
| small nuclear ribonucleoprotein Sm D1 | 32959908 | 6.5 | 141 |
| similar to Small nuclear ribonucleoprotein Sm D1 (Sm-D | 34877889 | 7 | 142 |
| autoantigen) | | | |
| FBRNP | 399758 | 5 | 143 |
| histone deacetylase 2 | 4557641 | 5.2 | 144 |
| huMCM5 | 1232079 | 3 | 145 |
| Histone H1b | 356168 | 6.5 | 146 |
| Histone macroH2A1.1 | 3493531 | 5.6 | 147 |
| Histone H2B.h (H2B/h) | 7387738 | 6.3 | 148 |
| HIST1H2BJ protein | 15680004 | 5 | 149 |
| H2A histone family, member Z; H2AZ histone | 4504255 | 6 | 150 |
| H2A histone family, member N | 4504249 | 6 | 151 |
| Histone H2A.5 | 70686 | 7.5 | 152 |
| similar to H3 histone, family 3B | 30156584 | 6.4 | 153 |
| H4 Histone family, member M | 4504321 | 8 | 154 |
| H4 Histone family, member L | 4504319 | 8 | 155 |
| H3 histone family, member T | 4504299 | 7 | 156 |
| H1 histone family, member 0 | 12652787 | 8 | 157 |
| histone H3 | 386772 | 8 | 158 |
| histone H4 | 223582 | 8 | 159 |
| heterochromatin-like protein 1 | 7416937 | 6.3 | 160 |
| inhibitor-2 of protein phosphatase-2A | 34604766 | 2.8 | 161 |
| UDP-glucose dehydrogenase | 18490087 | 3.3 | 162 |
| nucleolar protein 5A; | 32483374 | 3.6 | 163 |
| Nucleolar protein NOP5/NOP58 | 21595782 | 7.7 | 164 |
| nuclear RNA helicase | 1905998 | 3.8 | 165 |
| origin recognition complex subunit LATHEO | 5114107 | 3.2 | 166 |
| protein kinase, DNA-activated | 32140473 | 7 | 167 |
| PTB-associated splicing factor | 38458 | 8 | 168 |
| poly(ADP-ribose) polymerase | 190167 | 5 | 169 |
| proliferating cell nuclear protein P120 | 189422 | 2.5 | 170 |
| Proliferation associated cytokine-inducible protein CIP | 51094627 | 2.7 | 171 |
| Phosphoglycerate dehydrogenase | 15030035 | 3.3 | 172 |
| RNA helicase A - human | 1082769 | 3.7 | 173 |
| RNA helicase Gu - human (fragment) | 2135315 | 4 | 174 |
| RNA-binding region containing protein 2 isoform b; | 4757926 | 3.9 | 175 |
| replication licensing factor MCM5 - human | 2135871 | 3 | 176 |
| splicing factor 3b, subunit 1, 155 kDa; | 6912654 | 6 | 177 |
| splicing factor 3b, subunit 3, 130 kDa; | 40254849 | 5 | 178 |
| structure specific recognition protein 1; | 4507241 | 4.3 | 179 |
| SmB/B′ autoimmune antigene | 36495 | 9 | 180 |
| transketolase | 37267 | 3.1 | 181 |
| transcription factor NF-AT 90K chain | 1082856 | 3.2 | 182 |
| THOC4 protein | 30411083 | 5.4 | 183 |
| transformation upregulated nuclear protein | 460789 | 2.4 | 184 |
| ULIP | 1536909 | 2.4 | 185 |

Many upregulated proteins we identified have been reported as having increased expression in tumors. Expression of osteoblast specific factor 2 (OSF-2, periostin), a secreted matrix protein, increased 12 fold in breast carcinomas (Table 2). Our immunohistochemical studies on human breast tissue sections with normal or carcinomas suggest that OSF-2 is overexpressed in patients with breast carcinomas, in agreement with the result obtained from mass spectrometry. OSF-2 is overexpressed in the plasma membrane of breast cancer cells and, by a comparision between normal and tumor cells, OSF-2 appears to be translocated between the nuclear membrane and the plasma membrane in these two respective states.

TABLE 2
Proteins Having a Difference in Abundance of 3-Fold or Greater
between Breast Cancer Cells and Normal Cells

| | | SILAC | |
|---|---|---|---|
| | NCBI | quanti- | SEQ |
| | GI number | fication | ID NO |
| Downregulated proteins: | | | |
| Membrane or membrane-bound | | | |
| proteins | | | |
| Chain C, Annexin V | 809190 | 5.5 | 7 |
| CD81 antigen | 12804239 | 3.7 | 9 |
| 1-8D | 23396 | 3.4 | 13 |
| epididymal protein | 23092553 | 8.6 | 17 |
| glutaminase | 12044394 | 5.5 | 19 |
| hexokinase 1 isoform HKI | 4504391 | 3.5 | 22 |
| inter-alpha-trypsin inhibitor heavy chain | 3024064 | 5.3 | 25 |
| 3 precursor | | | |
| lysosome-associated membrane | 21070332 | 3.0 | 27 |
| protein-3 variant | | | |
| membrane-type matrix | 793763 | 4.3 | 28 |
| metalloproteinase | | | |
| macroglobulin alpha2 | 224053 | 7.5 | 29 |
| Membrane alanine aminopeptidase, | 37590640 | 5.0 | 30 |
| precursor | | | |
| membrane alanine aminopeptidase | 4502095 | 10.0 | 31 |
| precursor | | | |
| type-2 phosphatidic acid | 3015569 | 5.1 | 33 |
| phosphohydrolase | | | |
| pregnancy zone protein precursor | 131756 | 6.4 | 36 |
| plasminogen activator inhibitor type 1, | 24307907 | 3.8 | |
| member 2 | | | |
| transglutaminase 2 isoform a; | 39777597 | 6.5 | 42 |
| transglutaminase C | | | |
| Unknown | | | |
| unnamed protein product | 7023123 | 7.5 | 47 |
| Others | | | |
| 5′-nucleotidase | 23897 | 4.0 | 51 |
| Upregulated proteins: | | | |
| Membrane or membrane-bound | | | |
| proteins | | | |
| alpha 1 type III collagen; | 4502951 | 4.0 | 52 |
| alpha 3 type VI collagen isoform 3 | 55743102 | 3.2 | 54 |
| precursor; | | | |
| autoantigen p542 | 3334899 | 7.5 | 55 |
| apoptosis inhibitor 5 | 30583025 | 4.9 | 56 |
| Chain A, Synthetic Ubiquitin With | 31615803 | 3.7 | 59 |
| Fluoro-Leu At 50 And 67 | | | |
| Chain D, Tsg101(Uev) Domain In | 48425523 | 3.6 | 61 |
| Complex With Ubiquitin | | | |
| Carbamoylphosphate synthetase | 41351087 | 3.0 | 62 |
| 2/aspartate transcarbamylase | | | |
| cathepsin B | 3929733 | 3.0 | 63 |
| coproporphyrinogen oxidase | 433888 | 3.4 | 65 |
| cysteine protease | 1890050 | 3.3 | 66 |
| 4F2 heavy chain antigen | 177216 | 4.0 | 68 |
| farnesyl-diphosphate | 435677 | 3.3 | 70 |
| farnesyltransferase | | | |
| FBRL_HUMAN; 34 KD Nucleolar | 3399667 | 8.0 | 71 |
| Scleroderma antigen | 3153873 | 3.1 | 72 |
| putative G-binding protein | | | |
| integrin beta 4 binding protein isoform a | 4504771 | 3.4 | 73 |
| lysosomal proteinase cathepsin B | 181178 | 5.0 | 74 |
| matrin 3 | 6563246 | 4.5 | 75 |
| methylene tetrahydrofolate | 5729935 | 4.3 | 76 |
| dehydrogenase 2 precursor | | | |
| Osteoblast-specific factor 2 | 46576887 | 12.0 | 79 |
| Opa-interacting protein OIP2 | 2815604 | 4.5 | 81 |
| RAD50 homolog isoform 1 | 19924129 | 3.3 | 83 |
| SP-H antigen | 743447 | 5.0 | 84 |
| SWI/SNF-related matrix-associated | 18606276 | 4.5 | 85 |
| actin-dependent regulator | | | |
| scaffold attachment factor B | 1213639 | 3.2 | 88 |
| Solute carrier family 7 (cationic amino | 27503713 | 3.3 | 90 |
| acid transporter) | | | |
| thyroid-lupus autoantigen p70 | 4503841 | 5.0 | 91 |
| Unknown | | | |
| unnamed protein product | 21750187 | 4.0 | 95 |
| unnamed protein product | 32097 | 5.8 | 96 |
| unnamed protein product | 7022744 | 6.1 | 97 |
| unnamed protein product | 21749696 | 7.5 | 98 |
| unnamed protein product | 10434070 | 4.4 | 99 |
| unknown | 9789023 | 3.9 | 101 |
| hypothetical protein | 21739574 | 4.2 | 103 |
| hypothetical protein | 13276691 | 4.0 | 104 |
| hypothetical protein | 21739884 | 4.1 | 105 |
| nucleolar protein NOP5 (NOP58) | 17380155 | 7.5 | 106 |
| mago-nashi homolog (FLJ10292) | 15012020 | 6.3 | 107 |
| LAS1-like protein (FLJ12525) | 15778927 | 6.4 | 108 |
| Human mRNA, complete cds | 348239 | 7.5 | 109 |
| FTSJ3 protein [Homo sapiens] | 62914003 | 5.0 | 110 |
| PREDICTED: similar to RIKEN cDNA | 50745107 | 8.0 | 111 |
| 0610009D07 | | | |
| MGC2477 protein | 12655159 | 4.4 | 113 |
| Others | | | |
| B23 nucleophosmin | 825671 | 4.5 | 57 |
| chromosome segregation protein smc1 | 2135244 | 6.1 | 119 |
| CPSF6 protein | 12653847 | 6.2 | 123 |
| DDX17 protein | 12653635 | 4.2 | 125 |
| DNA-activated protein kinase, catalytic | 38258929 | 6.8 | 126 |
| subunit - human activating signal | | | |
| cointegrator 1 complex subunit 3-like 1 | 40217847 | 6.2 | 130 |
| hnRNA-binding protein M4 | 55977747 | 10.0 | 133 |
| similar to snRNP Sm D1 (Sm-D | 34877889 | 7.0 | 142 |
| autoantigen) | | | |
| histone deacetylase 2 | 4557641 | 5.2 | 144 |
| Histone H1b | 356168 | 6.5 | 146 |
| Histone H2A.5 | 70686 | 7.5 | 152 |
| similar to H3 histone, family 3B | 30156584 | 6.4 | 153 |
| histone H3 | 386772 | 8.0 | 158 |
| histone H4 | 223582 | 8.0 | 159 |
| Nucleolar protein NOP5/NOP58 | 21595782 | 7.7 | 164 |
| protein kinase, DNA-activated | 32140473 | 7.0 | 167 |
| PTB-associated splicing factor | 38458 | 8.0 | 168 |
| SmB/B′ autoimmune antigen | 36495 | 9.0 | 180 |
| Downregulated proteins: | | | |
| Membrane or membrane-bound | | | |
| proteins | | | |
| Chain C, Annexin V | 809190 | 5.5 | 7 |
| CD81 antigen | 12804239 | 3.7 | 9 |
| 1-8D | 23396 | 3.4 | 13 |
| epididymal protein | 23092553 | 8.6 | 17 |
| glutaminase | 12044394 | 5.5 | 19 |
| hexokinase 1 isoform HKI | 4504391 | 3.5 | 22 |
| inter-alpha-trypsin inhibitor heavy chain | 3024064 | 5.3 | 25 |
| 3 precursor | | | |
| lysosome-associated membrane | 21070332 | 3.0 | 27 |
| protein-3 variant | | | |
| membrane-type matrix | 793763 | 4.3 | 28 |
| metalloproteinase | | | |
| macroglobulin alpha2 | 224053 | 7.5 | 29 |
| Membrane alanine aminopeptidase, | 37590640 | 5.0 | 30 |
| precursor | | | |
| membrane alanine aminopeptidase | 4502095 | 10.0 | 31 |
| precursor | | | |
| type-2 phosphatidic acid | 3015569 | 5.1 | 33 |
| phosphohydrolase | | | |
| pregnancy zone protein precursor | 131756 | 6.4 | 36 |
| plasminogen activator inhibitor type 1, | 24307907 | 3.8 | |
| member 2 | | | |
| transglutaminase 2 isoform a; | 39777597 | 6.5 | 42 |
| transglutaminase C | | | |
| Unknown | | | |
| unnamed protein product | 7023123 | 7.5 | 47 |
| Others | | | |
| 5′-nucleotidase | 23897 | 4.0 | 51 |
| Upregulated proteins: | | | |
| Membrane or membrane-bound | | | |
| proteins | | | |
| alpha 1 type III collagen; | 4502951 | 4.0 | 52 |
| alpha 3 type VI collagen isoform 3 | 55743102 | 3.2 | 54 |
| precursor; | | | |
| autoantigen p542 | 3334899 | 7.5 | 55 |
| apoptosis inhibitor 5 | 30583025 | 4.9 | 56 |
| Chain A, Synthetic Ubiquitin With | 31615803 | 3.7 | 59 |
| Fluoro-Leu At 50 And 67 | | | |
| Chain D, Tsg101(Uev) Domain In | 48425523 | 3.6 | 61 |
| Complex With Ubiquitin | | | |
| Carbamoylphosphate synthetase | 41351087 | 3.0 | 62 |
| 2/aspartate transcarbamylase | | | |
| cathepsin B | 3929733 | 3.0 | 63 |
| coproporphyrinogen oxidase | 433888 | 3.4 | 65 |
| cysteine protease | 1890050 | 3.3 | 66 |
| 4F2 heavy chain antigen | 177216 | 4.0 | 68 |
| farnesyl-diphosphate | 435677 | 3.3 | 70 |
| farnesyltransferase | | | |
| FBRL_HUMAN; 34 KD Nucleolar | 3399667 | 8.0 | 71 |
| Scleroderma antigen | 3153873 | 3.1 | 72 |
| putative G-binding protein | | | |
| integrin beta 4 binding protein isoform a | 4504771 | 3.4 | 73 |
| lysosomal proteinase cathepsin B | 181178 | 5.0 | 74 |
| matrin 3 | 6563246 | 4.5 | 75 |
| methylene tetrahydrofolate | 5729935 | 4.3 | 76 |
| dehydrogenase 2 precursor | | | |
| Osteoblast-specific factor 2 | 46576887 | 12.0 | 79 |
| Opa-interacting protein OIP2 | 2815604 | 4.5 | 81 |
| RAD50 homolog isoform 1 | 19924129 | 3.3 | 83 |
| SP-H antigen | 743447 | 5.0 | 84 |
| SWI/SNF-related matrix-associated | 18606276 | 4.5 | 85 |
| actin-dependent regulator | | | |
| scaffold attachment factor B | 1213639 | 3.2 | 88 |
| Solute carrier family 7 (cationic amino | 27503713 | 3.3 | 90 |
| acid transporter) | | | |
| thyroid-lupus autoantigen p70 | 4503841 | 5.0 | 91 |
| Unknown | | | |
| unnamed protein product | 21750187 | 4.0 | 95 |
| unnamed protein product | 32097 | 5.8 | 96 |
| unnamed protein product | 7022744 | 6.1 | 97 |
| unnamed protein product | 21749696 | 7.5 | 98 |
| unnamed protein product | 10434070 | 4.4 | 99 |
| unknown | 9789023 | 3.9 | 101 |
| hypothetical protein | 21739574 | 4.2 | 103 |
| hypothetical protein | 13276691 | 4.0 | 104 |
| hypothetical protein | 21739884 | 4.1 | 105 |
| nucleolar protein NOP5 (NOP58) | 17380155 | 7.5 | 106 |
| mago-nashi homolog (FLJ10292) | 15012020 | 6.3 | 107 |
| LAS1-like protein (FLJ12525) | 15778927 | 6.4 | 108 |
| Human mRNA, complete cds | 348239 | 7.5 | 109 |
| FTSJ3 protein [Homo sapiens] | 62914003 | 5.0 | 110 |
| PREDICTED: similar to RIKEN cDNA | 50745107 | 8.0 | 111 |
| 0610009D07 | | | |
| MGC2477 protein | 12655159 | 4.4 | 113 |
| Others | | | |
| B23 nucleophosmin | 825671 | 4.5 | 57 |
| chromosome segregation protein smc1 | 2135244 | 6.1 | 119 |
| CPSF6 protein | 12653847 | 6.2 | 123 |
| DDX17 protein | 12653635 | 4.2 | 125 |
| | 38258929 | 6.8 | 126 |
| | 40217847 | 6.2 | 130 |
| | 55977747 | 10.0 | 133 |
| | 34877889 | 7.0 | 142 |
| | 4557641 | 5.2 | 144 |
| | 356168 | 6.5 | 146 |
| | 70686 | 7.5 | 152 |
| | 30156584 | 6.4 | 153 |
| | 386772 | 8.0 | 158 |
| | 223582 | 8.0 | 159 |
| | 21595782 | 7.7 | 164 |
| | 32140473 | 7.0 | 167 |
| | 38458 | 8.0 | 168 |
| | 36495 | 9.0 | 180 |

Further analysis of upregulated non-membrane proteins reveals that many are involved in cell cycle control, activation of chromatin, regulation of transcription, mRNA stability, and protein synthesis. For example, Cell cycle regulatory protein, DNA helicase, RNA helicase, DNA topoisomerase, DNA-activated protein kinase, histone deacetylase, cleavage and polyadenylation specific factor, splicing factor, heterogeneous nuclear ribonucleoprotein as well as histone proteins are significantly upregulated, which might be related to the rapid growth of cancer cells relative to normal cells.

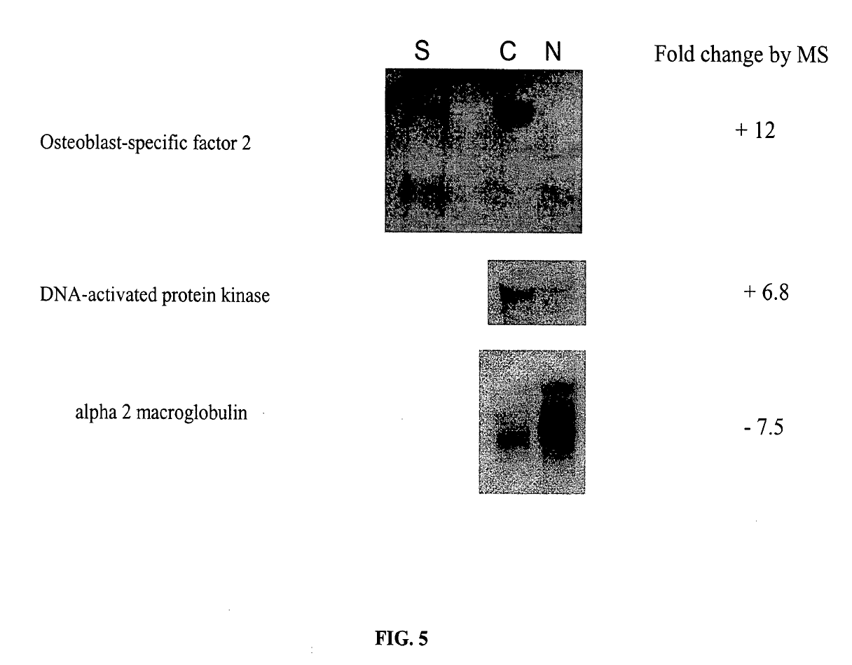

FIG. 5 depicts a Western blot of three of the proteins identified in this study as having differential expression in normal breast and breast cancer cells. Osteoblast specific factor 2 was demonstrated to have twelve-fold greater abundance in cancer cells than in normal cells by cell culture heavy label incorporation/mass spectrometry, and the Western blot that juxtaposes the cancerous (C) and normal (N) cell lysates is consistent with this result. (The protein is also detected in the media (S). DNA activated protein kinase also was found to be present in cancer cells at a higher level (6.8 fold) than in normal breast cells; this difference is also confirmed by Western blot. Alpha-2 macroglobulin, by contrast, was found to be of lower abundance in cancer cells when compared with normal cells by metabolic heavy isotope labeling in cell culture and mass spectrometry. This difference is again reflected in the Western blot of FIG. 5.

Using SILACIMS, we have established a high-throughput characterization of membrane proteins. A set of proteins chosen for further analysis by cell staining and Western blot were all verified as being differentially expressed in breast cancer and normal breast cells. Our results indicate that SILAC is a powerful technique for global identification and quantification of protein expression, and holds great promise in biomarker and drug target discovery efforts.

### Example 2

### Phosphopeptide Enrichment from Tryptic Digests Using Immobilized Metal Affinity Chromatography (imac) Resins

We have found that a mere two to four micro liters of Toyopearl AF-Chelate-650M resin material packed into an Eppendorf GeLoading tip, charged with 100 mM FeCl₃, and equilibrated with 0.1% acetic acid creates a microcolumn suitable for enrichment of as much as 30 pmole of phosphopeptides.

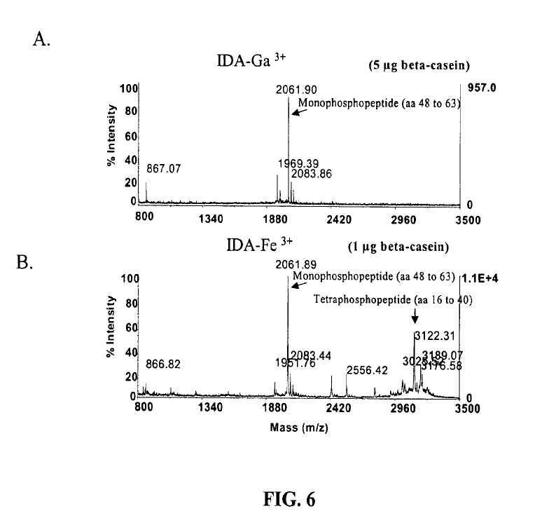

With tryptic digest of beta-casein as a test sample, we compared the quality of Toyopearl IDA resin charged with FeCl₃ with a commercially available phosphopeptide isolation kit from Pierce (cat#89853) that includes a gallium-chelated IDA-based resin. Phosphopeptides were isolated from a mixture of tryptic peptides by passing the solution slowly through the tip column. After washing with 0.1% acetic acid and then with 25% acetonitrile in 0.1% acetic acid, the phosphopeptides were cluted with 6 to 10 μl of ammonium bicarbonate (pH 9.0) The eluted phosphopeptides were dried in a Speed Vac and resuspended in 2 μl of 10% acetonitrile in 0.1% TFA or 0.1% formic acid for direct MS analysis, or desalted with a C₁₈ ziptip column and eluted with 75% acetonitrile in 0.1% TFA. As depicted in FIG. 6, the phosphopeptide isolation kit from Pierce captured the monophosphorylated peptide of m/z 2062 with 5 μg of β-casein tryptic digest (FIG. 6A). However, the Toyopearl AF-Chelate 650M resin (Tosoh Bioscience, Montgomery, Pa., 2005 part numbers 14475, 19800, and 14907) was able to enrich both the monophosphopeptide of m/z 2062 and the tetraphosphopeptide of m/z 3122.3 with only 1 μg of β-casein tryptic digest (FIG. 6B).

This result indicates that the Toyopearl AF-Chelate 650M resin exhibits higher sensitivity for phosphopeptides than the phosphopeptide isolation kit than other available resins for phosphoproteins or phosphopeptides. To further test the ability of Toyopearl AF-Chelate 650M resin in the enrichment of phosphopeptides, Dok-2 protein band was excised from NuPAGE and in-gel digested with trypsin, the phosphopeptide was isolated from the tryptic digest as described above. As depicted in FIG. 7A, a peptide of m/z 1688 was clearly observed. Peptide sequence analysis indicated that this peptide corresponded to a phosphopeptide of Dok-2 with tyrosine phosphorylated at residue 299 (SEQ ID NO:186) (FIG. 7B). This result indicates that the Toyopearl AF-Chelate 650M resin works on samples of biological origin.

### Example 3

SILAC is a also powerful tool for quantifying posttranslational modifications. We examined the change in the level of phosphosphorylation of Dok-2 in response to treatment of ST1571, an inhibitor of Bcr-Abl kinase, in human chronic myelogenous leukemia cells. As a control, aliquots of heavy tyrosine (U-¹³C₉) labeled cells (5×10⁸) were mixed equally with light tyrosine (¹²C) labeled cells (5×10⁸) before the drug treatment.

A first aliquot of heavy tyrosine labeled cells (5×10⁸) was preincubated with 1 μM of STI571 at 37° C. for 1 h and then mixed with an equal number of light (natural abundance) isotope tyrosine labeled cells. A second aliquot of heavy tyrosine labeled cells (5×10⁸) was preincubated with 1 μM of ST1571 at 37° C. for 2 h and then also mixed with an equal number of light (natural abundance) isotope tyrosine labeled cells. In both cases, the cell mixtures (1×10⁹) were lysed with NP40 lysis buffer and incubated with 30 μg of the GST fusion of the SH₂ domain of SHIP1 conjugated to glutathione Sepharose 4B. Affinity captured proteins were then analyzed by SDS-PAGE on a NuPAGE® acrylamide gel (Invitrogen, Carlsbad, Calif.) and stained with SimplyBlue™ SafeStain™ protein gel stain (Invitrogen, Carlsbad, Calif.). The Dok-2 protein bands from each mixed cell sample were excised and digested with trypsin. The resulting tryptic peptides were analyzed by MALDI-TOF.

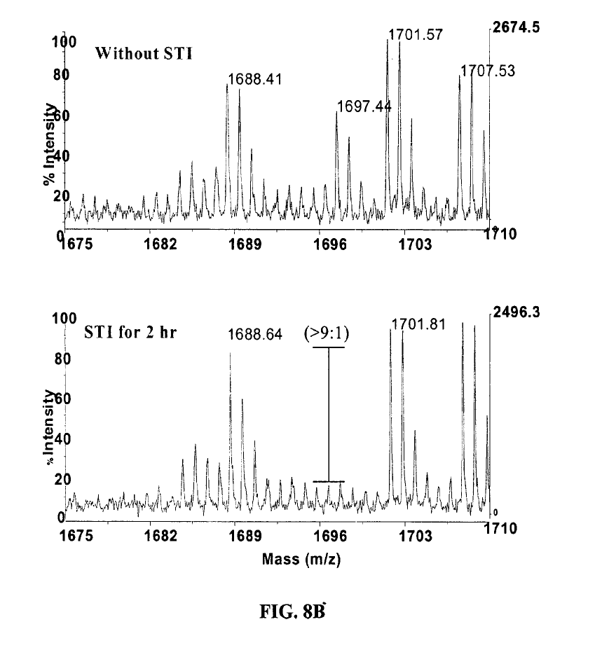

As depicted in FIG. 8A, when CML cells were incubated with ST1571 for 1 h, the amount of phosphopeptide of Dok2 reduced to 75%, which was calculated from the peptide ratio of m/z 1688 to m/z 1697. When cells were incubated with ST1571 for 2 h, the heavy labeled phosphopeptide of 1697 was inhibited more than 90%, as quantitated from the ratio of isotopic peptide pairs (FIG. 8B). This result indicates that quantitation that the phosphoproteomics methods presented herein can be targeted to specific transduction pathways, by studying phosphorylation cascades in Dok2/p210bcr/abl-expressing hematopoietic cells.

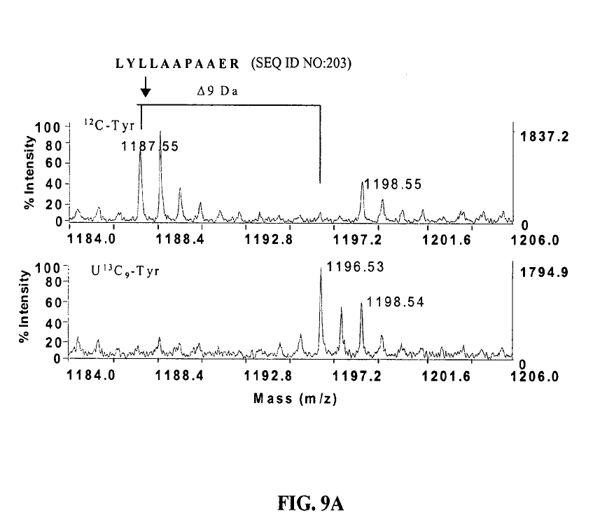

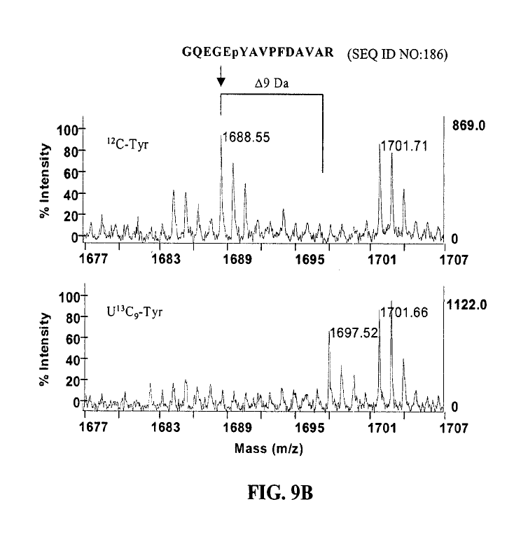

Based on metabolic labeling of proteins with light and heavy tyrosine, and using affinity capture reagents for SHIP2 and Dok2, we were able to determine the change in phosphorylation of Bcr-Abl kinase, as well as its downstream substrates SHIP2 and Dok2, in response to ST1571 treatment in human chronic myelogenous leukemia (CML) cells. In this study, we have identified two phosphopeptides from Bcr-Abl kinase, one from SHIP2 (FIG. 9A), and one from Dok-2 (FIG. 9B). Dok-2 is a substrate of Bcr-Abl kinase, which displays increased tyrosine phosphorylation levels in primary CML progenitor cells in human patients. We identified a novel phosphorylation site in Dok-2 (Tryosine 299 in tryptic peptide GQEGEYAVPFDAVAR (SEQ ID NO:186)) as shown in FIG. 9B, and have shown that the phosphorylation level of this site is decreased 75% upon 1 h treatment with ST1571 and was inhibited more than 90% upon treatment with STI571 for 2 h. Table 3 shows phosphopeptides found in increased abundance in non-treated CML cells as compared with ST1571-treated CML cells.

TABLE 3
Phosphopeptides in CML Cells

| | | |
|---|---|---|
| Name of Protein | Genbank gi | Peptide Sequence |

| | | |
|---|---|---|
| BCR-ABL Kinase | 514267 | LMTGDTpyTAHAGAK |
| | | (SEQ ID NO: 200) |
| | 487345 | NSLETLLpYKPVDR |
| | | (SEQ ID NO: 201) |
| Caspase recruit- | 17977658 | DENYMIAMR |
| ment domain | | (SEQ ID NO: 202) |
| protein 10 | | |
| Dok-2 protein | 41406050 | GQEGEpYAVPFDAVAR |
| | | (SEQ ID NO: 186) |
| | | LYLLAAPAAER |
| | | (SEQ ID NO: 203) |
| FERM and PDZ | 662416 | DLACLIAGYYR |
| domain contain- | | (SEQ ID NO: 204) |
| ing 1 | | |
| GRB-2associated | 6912460 | MSGDPDLEYYK |
| binding 2 iso- | | (SEQ ID NO: 205) |
| form b | | |
| SHC | 284403 | ESTTTPGQYVLTGLQSGQPK |
| | | (SEQ ID NO: 206) |
| SHIP2 | 4755142 | TLSEVDpYAPAGPAR |
| | | (SEQ ID NO: 207) |
| SHIP1 | 1888525 | AIQDYLSTQLAQDSEFVK |
| | | (SEQ ID NO: 208) |

This approach is applicable to the study of regulation of phosphorylation in response to stimuli or drug treatment in cell culture in general.

Although the invention has been described with reference to the above examples, it will be understood that modifications and variations are encompassed within the spirit and scope of the invention.

All headings are for the convenience of the reader, and are not intended to limit the scope of the invention.

All references cited herein, including patents, patent applications, and publications, are incorporated by reference in their entireties.

1-131. (canceled)

132. A method of detecting one or more biomolecules, comprising detecting in a biological sample, expression of a protein of Table 1, or a nucleic acid encoding a protein of Table 1, wherein said biological sample is a sample of a patient with a breast pathology.

133. The method of claim 132, wherein said biological sample is a tumor biopsy sample.

134. The method of claim 132, wherein said biological sample is a breast tumor biopsy sample.

135. The method of claim 132, wherein the biological sample is a serum sample.

136. The method of claim 132, wherein said biological sample comprises blood, plasma, serum, urine, saliva, lymphatic fluid, pelvic lavage, lung aspirate, nipple aspirate, or breast duct lavage.

137. The method of claim 132, wherein expression is detected for two or more proteins of Table 1, or nucleic acids encoding two or more proteins of Table 1.

138. The method of claim 132, wherein expression is detected for three or more proteins of Table 1, or nucleic acids encoding three or more proteins of Table 1.

139. The method of claim 132, wherein expression is detected for four or more proteins of Table 1, or nucleic acids encoding four or more proteins of Table 1.

140. The method of claim 132, wherein expression is detected for one or more proteins of Table 2, or nucleic acids encoding one or more proteins of Table 2.

141. The method of claim 132, wherein the protein is annexin V chain c (SEQ ID NO:7), epididymal protein (SEQ ID NO:17), glutaminase (SEQ ID NO:19), inter-alpha-trypsin inhibitor heavy chain 3 precursor (SEQ ID NO:25), alpha 2 macroglobulin (SEQ ID NO:29), membrane alanine aminopeptidase precursor (SEQ ID NO:30), type-2 phosphatidic acid phosphohydrolase (SEQ ID NO:33), pregnancy zone protein precursor (SEQ ID NO:36), transglutaminase 2 isoform a/transglutaminase C (SEQ ID NO:42), the unnamed protein product having a sequence with NCBI database Genbank gi 7023123 (SEQ ID NO:47), autoantigen p542 (SEQ ID NO:55), the 34 kD nucleolar sclerodema antigen (SEQ ID NO:71), lysosomal proteinase cathepsin b (SEQ ID NO:74), osteoblast-specific factor 2 (SEQ ID NO:79), SP—H antigen (SEQ ID NO:84), thyroid-lupus autoantigen p70 (SEQ ID NO:91), the unnamed protein product having NCBI database Genbank gi 32097 (SEQ ID NO:96), the unnamed protein product having NCBI database Genbank gi 7022744 (SEQ ID NO:97), the unnamed protein product having NCBI database Genbank gi 21749696 (SEQ ID NO:98), a hypothetical protein (mago-nashi homolog) having NCBI database Genbank gi 15012020 (SEQ ID NO:107), a hypothetical protein (LS1-like) having NCBI database Genbank gi 15778927 (SEQ ID NO:108), a protein (human mR_NA, complete cds gene product) having NCBI database Genbank gi 348239 (SEQ ID NO:109), FTSJ3 protein (SEQ ID NO:110), a predicted protein (similar to RIKEN cDNA 0610009D07) having NCBI having database Genbank gi 50745107(SEQ ID NO:111), chromosome segregation protein smc1 (SEQ ID NO:119), CPSF6 protein (SEQ ID NO:123), DNA-activated protein kinase (SEQ ID NO:167) and DNA-activated protein kinase catalytic subunit (SEQ ID NO:126), activating signal cointegrator 1 complex subunit 3-like 1 (SEQ ID NO:130), heterogeneous nuclear ribonucleoprotein M, (SEQ ID NO:133) a protein similar to small nuclear ribonucleoprotein D1 having NCBI database Genbank gi 34877889 (SEQ ID NO:142), histone deacetylase 2 (SEQ ID NO:144), histone Hlb (SEQ ID NO:146), histone H2A.5 (SEQ ID NO:152), a protein similar to histone H3 having NCBI database Genbank gi 30156584 (SEQ ID NO:153), histone H3 (SEQ ID NO:158), histone H4 (SEQ ID NO:159), nucleolar protein NOP51NOP58 (SEQ ID NO:107; SEQ ID NO:164), PTB-associated splicing factor (SEQ ID NO:168), or SmB/B′ autoimmune antigen (SEQ ID NO:180).

142. The method of claim 132, wherein the protein is annexin V chain c (SEQ ID NO:7), epididymal protein (SEQ ID NO:17), glutaminase (SEQ ID NO:19), inter-alpha-trypsin inhibitor heavy chain 3 precursor (SEQ ID NO:30), alpha 2 macroglobulin (SEQ ID NO:29), membrane alanine aminopeptidase precursor (SEQ ID NO:31), type-2 phosphatidic acid phosphohydrolase (SEQ ID NO:33), pregnancy zone protein precursor (SEQ ID NO:36), transglutaminase 2 isoform a/transglutaminase C (SEQ ID NO:42), or the unnamed protein product having a sequence with NCBI database Genbank gi 7023123 (SEQ ID NO:47).

143. The method of claim 132, wherein the protein is autoantigen p542 (SEQ ID NO:55), the 34 kD nucleolar sclerodema antigen (SEQ ID NO:71), lysosomal proteinase cathepsin b (SEQ ID NO:74), osteoblast-specific factor 2 (SEQ ID NO: 79), SP—H antigen (SEQ ID NO:84), thyroid-lupus autoantigen p70 (SEQ ID NO:9), the unnamed protein product having NCBI database Genbank gi 32097 (SEQ ID NO:96), the unnamed protein product having NCBI database Genbank gi 7022744 (SEQ ID NO:97), the unnamed protein product having NCBI database Genbank gi 21749696 (SEQ ID NO:98), a hypothetical protein (mago-nashi homolog) having NCBI database Genbank gi 15012020 (SEQ ID NO:107), a hypothetical protein (LS1-like) having NCBI database Genbank gi 15778927 (SEQ ID NO:108), a protein (human mRNA, complete cds gene product) having NCBI database Genbank gi 348239 (SEQ ID NO:109), FTSJ3 protein (SEQ ID NO:110), a predicted protein (similar to RIKEN cDNA 0610009D07) having NCBI having database Genbank gi 50745107 (SEQ ID NO:111), chromosome segregation protein smc 1 (SEQ ID NO:119; Genbank gi 2135244), CPSF6 protein (SEQ ID NO:123), DNA-activated protein kinase (SEQ ID NO:167) and DNA-activated protein kinase catalytic subunit (SEQ ID NO:126), activating signal cointegrator 1 complex subunit 3-like 1 (SEQ ID NO:130), heterogeneous nuclear ribonucleoprotein M (SEQ ID NO:133), a protein similar to small nuclear ribonucleoprotein D1 having NCBI database Genbank gi 34877889 (SEQ ID NO:142), histone deacetylase 2 (SEQ ID NO:144), histone H1b ((SEQ ID NO:146), histone H2A.5 (SEQ ID NO:152), a protein similar to histone H3 having NCBI database Genbank gi 30156584 (SEQ ID NO:153), histone H3 (SEQ ID NO:158), histone H4 (SEQ ID NO:159), nucleolar protein NOP5/NOP58 (SEQ ID NO:162), PTB-associated splicing factor (SEQ ID NO:168), or SmB/B′ autoimmune antigen (SEQ ID NO:180).

144. The method of claim 132, wherein an expression level of a protein of Table 1, or a nucleic acid encoding a protein of Table 1 is determined.

145. The method of claim 144, wherein an altered expression level in the biological sample compared to a normal sample is indicative of the presence of a breast pathology.

146. The method of claim 144, wherein an altered expression level in the biological sample compared to a normal sample is indicative of the presence of breast cancer.

147. The method of claim 144, further comprising correlating the expression level of the protein or the nucleic acid with a type of cancer.

148. The method of claim 144, further comprising correlating the expression level of said protein or nucleic acid with a stage of cancer.

149. The method of claim 144, further comprising correlating the expression level of said protein or nucleic acid with a prognosis.

150. The method of claim 144, further comprising correlating the expression level of said protein or nucleic acid with response to one or more anti-cancer agents.

151. The method of claim 132, wherein the detecting comprising contacting the biological sample with a specific binding reagent that binds to the protein or the nucleic acid.

152. The method of claim 151, wherein the specific binding reagent is an antibody or a nucleic acid.

153. The method of claim 132, wherein the detecting comprises an immunoassay.

154. A kit comprising a specific binding reagent that binds to a protein of Table 1, or that binds to a nucleic acid encoding a protein of Table 1, and a control, wherein the control is a biological sample derived from a subject having a breast pathology.

155. The kit of claim 154, wherein the specific binding reagent is an antibody or a nucleic acid.

156. The kit of claim 154, further comprising a specific binding reagent that binds to a second protein of Table 1, or that binds to a nucleic acid encoding a second protein of Table 1.

157. The kit of claim 154, further comprising a specific binding reagent that binds to a protein of Table 2, or that binds to a nucleic acid encoding a protein of Table 2.

158. The kit of claim 154, wherein the specific binding reagent binds to annexin V, chain c, epididymal protein, glutaminase, inter-alphatrypsin inhibiot heavy chain 3 precursor, alpha 2 macroglobulin, membrane alanine aminopeptidase, type-2 phosphatidic acid phosphohydrolase, pregnancy zone protein precursor (Genbank gi 131756), transglutaminase 2 isoform a/transglutaminase C, the unnamed protein product having a sequence with NCBI database Genbank gi 7023123, autoantigen p542, the 34 kD nucleolar sclerodema antigen, lysosomal proteinase cathepsin b, osteoblastspecific factor 2, SP—H antigen, thyroid-lupus autoantigen p70, the unnamed protein product having NCBI database Genbank gi 32097, the unnamed protein product having NCBI database Genbank gi 7022744, the unnamed protein product having NCBI database Genbank gi 21749696, a hypothetical protein having NCBI database Genbank gi 7512749, a hypothetical protein having NCBI database Genbank gi15012020, a hypothetical protein having NCBI database Genbank gi 15778927, a protein having NCBI database Genbank gi 348239, FTSJ3 protein, a protein having NCBI database Genbank gi 50745107, chromosome segregation protein smc 1, CPSF6 protein, DNA-activated protein kinase, U5 snRNP Sm D1(Sm-D autoantigen), histone deacetylase 2, histone H1b, histon H2A.5, histone H3, a protein similar to histone H3 having NCBI database Genbank gi 30156584, histone H4, nucleolar protein NOP5/NOP58, PTBassociated splicing factor, or SmB/B′ autoimmune antigen.

159. The kit of claim 154, wherein the specific binding reagent binds to a nucleic acid encoding annexin V, chain c, epididymal protein, glutaminase, inter-alpha-trypsin inhibiot heavy chain 3 precursor, alpha 2 macroglobulin, membrane alanine aminopeptidase, type-2 phosphatidic acid phosphohydrolase, pregnancy zone protein precursor (Genbank gi 131756), transglutaminase 2 isoform a/transglutaminase C, the unnamed protein product having a sequence with NCBI database Genbank gi 7023123, autoantigen p542, the 34 kD nucleolar sclerodema antigen, lysosomal proteinase cathepsin b, osteoblastspecific factor 2, SP—H antigen, thyroid-lupus autoantigen p70, the unnamed protein product having NCBI database Genbank gi 32097, the unnamed protein product having NCBI database Genbank gi 7022744, the unnamed protein product having NCBI database Genbank gi 21749696, a hypothetical protein having NCBI database Genbank gi 7512749, a hypothetical protein having NCBI database Genbank gi5012020, a hypothetical protein having NCBI database Genbank gi 15778927, a protein having NCBI database Genbank gi 348239, FTSJ3 protein, a protein having NCBI database Genbank gi 50745107, chromosome segregation protein smc1, CPSF6 protein, DNA-activated protein kinase, U5 snRNP Sm D1(Sm-D autoantigen), histone deacetylase 2, histone H1b, histon H2A.5, histone H3, a protein similar to histone H3 having NCBI database Genbank gi 30156584, histone H4, nucleolar protein NOP5/NOP58, PTBassociated splicing factor, or SmB/B′ autoimmune antigen.
